SEQUENCE LISTING (25 sequences)

SEQ ID NO: 1 - DNA, 11 bp, Artificial Sequence
  synthetic oligonucleotide
cgcagtcagcc

SEQ ID NO: 2 - DNA, 11 bp, Artificial Sequence
  synthetic oligonucleotide
ggctgcctgcg

SEQ ID NO: 3 - DNA, 11 bp, Artificial Sequence
  synthetic oligonucleotide
ggctgactgcg

SEQ ID NO: 4 - DNA, 11 bp, Artificial Sequence
  synthetic oligonucleotide
cgcagccagcc

SEQ ID NO: 5 - DNA, 11 bp, Artificial Sequence
  synthetic oligonucleotide
ggctggctgcg

SEQ ID NO: 6 - DNA, 11 bp, Artificial Sequence
  synthetic oligonucleotide
cgcaggcagcc

SEQ ID NO: 7 - DNA, 11 bp, Artificial Sequence
  synthetic oligonucleotide
ggctgtctgcg

SEQ ID NO: 8 - DNA, 11 bp, Artificial Sequence
  synthetic oligonucleotide
cgcagacagcc

SEQ ID NO: 9 - DNA, 11 bp, Artificial Sequence
  synthetic oligonucleotide
ggctgcatgcg

SEQ ID NO: 10 - DNA, 22 bp, Artificial Sequence
  synthetic oligonucleotide
cacgcattgcccgcgttgcgca

SEQ ID NO: 11 - DNA, 22 bp, Artificial Sequence
  synthetic oligonucleotide
tgcgcaacgcgtgcaatgcgtg

SEQ ID NO: 12 - DNA, 22 bp, Artificial Sequence
  synthetic oligonucleotide
tgcgcaacgcgggcaatgcgtg

SEQ ID NO: 13 - DNA, 38 bp, Artificial Sequence
  synthetic oligonucleotide
ggaagaaggcatacgggttaactagggcagcggacaat

SEQ ID NO: 14 - DNA, 38 bp, Artificial Sequence
  synthetic oligonucleotide
attgtccgctgccctagttcacccgtatgccttcttcc

SEQ ID NO: 15 - DNA, 38 bp, Artificial Sequence
  synthetic oligonucleotide
ggaagaaggcatacgggtgaactagggcagcggacaat

SEQ ID NO: 16 - DNA, 547 bp, Mus musculus
gcacgcgctggacgcgcatcgattccgtagagccacaccctgaagggccaatctgctcacacaggatagagagggcaggagccagggcagagcatataaggtgaggtaggatcagttgctcctcacatttgcttctgacatagttgtgttgactcacaaccccagaaacagacatcatggtgcacctgactgatgctgagaaggctgctgtctcttgcctgtggggaaaggtgaactccgatgaagttggtggtgaggccctgggcaggttggtatccaggttacaaggcagctcacaagaagaagttgggtgcttggagacagaggtctgctttccagcagacactaactttcagtgtcccctgtctatgtttccctttttaggctgctgttgtctacccttggacccagcggtactttgatagctttggagacctatcctctgcctctgctatcatgggtaatgccaaagtgaaggcccatggcaagaaggtgataactgcctttaacgatggcctgaatcacttggacagcctcaagggcacct

SEQ ID NO: 17 - DNA, 547 bp, Mus musculus
aggtgcccttgaggctgtccaagtgattcaggccatcgttaaaggcagttatcaccttcttgccatgggccttcactttggcattacccatgatagcagaggcagaggataggtctccaaagctatcaaagtaccgctgggtccaagggtagacaacagcagcctaaaaagggaaacatagacaggggacactgaaagttagtgtctgctggaaagcagacctctgtctccaagcacccaacttcttcttgtgagctgccttgtaacctggataccaacctgcccagggcctcaccaccaacttcatcggagttcacctttccccacaggcaagagacagcagccttctcagcatcagtcaggtgcaccatgatgtctgtttctggggttgtgagtcaacacaactatgtcagaagcaaatgtgaggagcaactgatcctacctcaccttatatgctctgccctggctcctgccctctctatcctgtgtgagcagattggcccttcagggtgtggctctacggaatcgatgcgcgtccagcgcgtgc

SEQ ID NO: 18 - DNA, 547 bp, Mus musculus
gcacgcgctggacgcgcatcgattccgtagagccacaccctgaagggccaatctgctcacacaggatagagagggcaggagccagggcagagcatataaggtgaggcaggatcagttgctcctcacatttgcttctgacatagttgtgttgactcacaaccccagaaacagacatcatggtgcacctgactgatgctgagaaggctgctgtctcttgcctgtggggaaaggtgaactccgatgaagttggtggtgaggccctgggcaggttggtatccaggttacaaggcagctcacaagaagaagttgggtgcttggagacagaggtctgctttccagcagacactaactttcagtgtcccctgtctatgtttccctttttaggctgctgttgtctacccttggacccagcggtactttgatagctttggagacctatcctctgcctctgctatcatgggtaatgccaaagtgaaggcccatggcaagaaggtgataactgcctttaacgatggcctgaatcacttggacagcctcaagggcacct

SEQ ID NO: 19 - DNA, 547 bp, Mus musculus
aggtgcccttgaggctgtccaagtgattcaggccatcgttaaaggcagttatcaccttcttgccatgggccttcactttggcattacccatgatagcagaggcagaggataggtctccaaagctatcaaagtaccgctgggtccaagggtagacaacagcagcctaaaaagggaaacatagacaggggacactgaaagttagtgtctgctggaaagcagacctctgtctccaagcacccaacttcttcttgtgagctgccttgtaacctggataccaacctgcccagggcctcaccaccaacttcatcggagttcacctttccccacaggcaagagacagcagccttctcagcatcagtcaggtgcaccatgatgtctgtttctggggttgtgagtcaacacaactatgtcagaagcaaatgtgaggagcaactgatcctgcctcaccttatatgctctgccctggctcctgccctctctatcctgtgtgagcagattggcccttcagggtgtggctctacggaatcgatgcgcgtccagcgcgtgc

SEQ ID NO: 20 - DNA, 547 bp, Mus musculus
gcacgcgctggacgcgcatcgattccgtagagccacaccctgaagggccaatctgctcacacaggatagagagggcaggagacagggcagagcatataaggtgaggtaggatcagttgctcctcacatttgcttctgacatagttgtgttgactcacaaccccagaaacagacatcatggtgcacctgactgatgctgagaaggctgctgtctcttgcctgtggggaaaggtgaactccgatgaagttggtggtgaggccctgggcaggttggtatccaggttacaaggcagctcacaagaagaagttgggtgcttggagacagaggtctgctttccagcagacactaactttcagtgtcccctgtctatgtttccctttttaggctgctgttgtctacccttggacccagcggtactttgatagctttggagacctatcctctgcctctgctatcatgggtaatgccaaagtgaaggcccatggcaagaaggtgataactgcctttaacgatggcctgaatcacttggacagcctcaagggcacct

SEQ ID NO: 21 - DNA, 547 bp, Mus musculus
aggtgcccttgaggctgtccaagtgattcaggccatcgttaaaggcagttatcaccttcttgccatgggccttcactttggcattacccatgatagcagaggcagaggataggtctccaaagctatcaaagtaccgctgggtccaagggtagacaacagcagcctaaaaagggaaacatagacaggggacactgaaagttagtgtctgctggaaagcagacctctgtctccaagcacccaacttcttcttgtgagctgccttgtaacctggataccaacctgcccagggcctcaccaccaacttcatcggagttcacctttccccacaggcaagagacagcagccttctcagcatcagtcaggtgcaccatgatgtctgtttctggggttgtgagtcaacacaactatgtcagaagcaaatgtgaggagcaactgatcctacctcaccttatatgctctgccctgtctcctgccctctctatcctgtgtgagcagattggcccttcagggtgtggctctacggaatcgatgcgcgtccagcgcgtgc

SEQ ID NO: 22 - DNA, 547 bp, Mus musculus
gcacgcgctggacgcgcatcgattccgtagagccacaccctgaagggccaatctgctcacacaggatagagagggcaggagcgagggcagagcatataaggtgaggtaggatcagttgctcctcacatttgcttctgacatagttgtgttgactcacaaccccagaaacagacatcatggtgcacctgactgatgctgagaaggctgctgtctcttgcctgtggggaaaggtgaactccgatgaagttggtggtgaggccctgggcaggttggtatccaggttacaaggcagctcacaagaagaagttgggtgcttggagacagaggtctgctttccagcagacactaactttcagtgtcccctgtctatgtttccctttttaggctgctgttgtctacccttggacccagcggtactttgatagctttggagacctatcctctgcctctgctatcatgggtaatgccaaagtgaaggcccatggcaagaaggtgataactgcctttaacgatggcctgaatcacttggacagcctcaagggcacct

SEQ ID NO: 23 - DNA, 547 bp, Mus musculus
aggtgcccttgaggctgtccaagtgattcaggccatcgttaaaggcagttatcaccttcttgccatgggccttcactttggcattacccatgatagcagaggcagaggataggtctccaaagctatcaaagtaccgctgggtccaagggtagacaacagcagcctaaaaagggaaacatagacaggggacactgaaagttagtgtctgctggaaagcagacctctgtctccaagcacccaacttcttcttgtgagctgccttgtaacctggataccaacctgcccagggcctcaccaccaacttcatcggagttcacctttccccacaggcaagagacagcagccttctcagcatcagtcaggtgcaccatgatgtctgtttctggggttgtgagtcaacacaactatgtcagaagcaaatgtgaggagcaactgatcctacctcaccttatatgctctgccctcgctcctgccctctctatcctgtgtgagcagattggcccttcagggtgtggctctacggaatcgatgcgcgtccagcgcgtgc

SEQ ID NO: 24 - DNA, 547 bp, Mus musculus
gcacgcgctggacgcgcatcgattccgtagagccacaccctgaagggccaatctgctcacacaggatagagagggcaggagccagggcagagcatataaggtgaggtaggatcagttgctccacacatttgcttctgacatagttgtgttgactcacaaccccagaaacagacatcatggtgcacctgactgatgctgagaaggctgctgtctcttgcctgtggggaaaggtgaactccgatgaagttggtggtgaggccctgggcaggttggtatccaggttacaaggcagctcacaagaagaagttgggtgcttggagacagaggtctgctttccagcagacactaactttcagtgtcccctgtctatgtttccctttttaggctgctgttgtctacccttggacccagcggtactttgatagctttggagacctatcctctgcctctgctatcatgggtaatgccaaagtgaaggcccatggcaagaaggtgataactgcctttaacgatggcctgaatcacttggacagcctcaagggcacct

SEQ ID NO: 25 - DNA, 547 bp, Mus musculus
aggtgcccttgaggctgtccaagtgattcaggccatcgttaaaggcagttatcaccttcttgccatgggccttcactttggcattacccatgatagcagaggcagaggataggtctccaaagctatcaaagtaccgctgggtccaagggtagacaacagcagcctaaaaagggaaacatagacaggggacactgaaagttagtgtctgctggaaagcagacctctgtctccaagcacccaacttcttcttgtgagctgccttgtaacctggataccaacctgcccagggcctcaccaccaacttcatcggagttcacctttccccacaggcaagagacagcagccttctcagcatcagtcaggtgcaccatgatgtctgtttctggggttgtgagtcaacacaactatgtcagaagcaaatgtgtggagcaactgatcctacctcaccttatatgctctgccctggctcctgccctctctatcctgtgtgagcagattggcccttcagggtgtggctctacggaatcgatgcgcgtccagcgcgtgc

(19) United States
(12) Patent Application Publication
(10) Pub. No.: US 2004/0146875 A1
(43) Pub. Date: Jul. 29, 2004

(54) Method of identifying differences between nucleic acid molecules

(76) Inventors: Richard Graham Hay Cotton, Kooyong Victoria (AU); Chinh Thien BUI, Footscray Victoria (AU); Andreana Lambrinakos, Lower Templestowe, Victoria (AU)

Correspondence Address: Greenlee Winner & Sullivan, Suite 201, 5370 Manhattan Circle, Boulder, CO, 80303, US

(21) Appl. No.: 10/468,671

(22) PCT Filed: Feb. 19, 2002
(86) PCT No.: PCT/AU02/00171

(30) Foreign Application Priority Data
Feb. 19, 2001 (AU) PR 3205
Mar. 20, 2001 (AU) PR 3855

(51) Int. Cl. C12Q 1/68

(52) U.S. Cl. 435/6

(57) ABSTRACT

The present invention relates generally to a method of identifying differences between nucleic acid molecules and provides a method capable of identifying a variation in a nucleotide sequence in nucleic acid molecules based on the selectivity of oxidizing agents towards mismatched or unmatched bases in a nucleic acid duplex. The method of the present invention enables the detection of nucleotide variations, such as resulting from base changes (mutations and polymorphisms) in target nucleic acid heteroduplex molecules derived from wildtype and mutant homoduplexes

### FIELD OF THE INVENTION

The present invention relates generally to a method of identifying differences between nucleic acid molecules. More particularly, the present invention provides a method capable of identifying a variation in a nucleotide sequence in nucleic acid molecules based on the selectivity of oxidizing agents towards mismatched or unmatched bases in a nucleic acid duplex compared with matched bases. The method of the present invention enables the detection of nucleotide variations, such as resulting from base changes (mutations and polymorphisms) in target nucleic acid heteroduplex molecules derived from wildtype and mutant homoduplexes.

### BACKGROUND OF THE INVENTION

The rapidly increasing sophistication of recombinant technology is greatly facilitationg research and development in all biological systems in regard to both control and regulation of gene activity (genomics), and an understanding of how gene function determines gene operations that control the synthesis of intra and extracellular cell components especially proteins and glycoproteins (proteomics) and cell surface receptors and other organelles and structures residing in or across the cell membranes.

Recombinant DNA/RNA technology has become a central tool in research to understand how individual cells, organs and whole organisms are created, grow to maturity and survive during their normal lifespan. Such capacity includes a knowledge of how they are able to react to repair and maintain their integrity in the face of physical, dietary, chemical, biological and other events that may occur which would otherwise lead to loss of physical integrity or even cell death.

This knowledge has also permitted the construction of unique biological and other entities which do not occur naturally in nature, including transgenic cells lines, animals, plants or other species that possess properties desirable for clinical and basic research, commercial, scientific, pharmaceutical, industrial or military applications.

The possibility of the creation of non-living DNA/RNA constructs for commercial or other activities such as use in industrial processes, scientific research, or military purposes also exists as a result of current knowledge.

Thus the ability to detect alterations in nucleotide sequences is now an important technical capability that permits a variety of scientific activities including but not limited to the following processes/events:

1) Confirming the normality of the genetic code of species including human, animal, plant bacterial viral, fungal, protozoan and other DNA/RNA based life forms and/or the absence of genetic manipulation of such life forms;

2) Detecting genetic abnormalities as a means of enabling diagnosis, prognosis, choice of optimal therapy in disease states, minimising adverse or toxic effects from therapeutic drugs and predicting or detecting disease recurrence and/or the evolution of disease processes during the course of an illness;

3) Allowing the precise breeding of animals including the creation of ‘new’ species which contain and/or express new genes that have been inserted to generate specific unique products or abilities for research, commercial or pharmaceutical purposes;

4) Have specific genes deleted from their genome (knockouts) for research or commercial purposes;

5) Allowing the precise breeding of plants including the creation of ‘new’ species which contain and/or express new genes that have been inserted to generate specific unique products or abilities for research, commercial or pharmaceutical purposes;

6) Have specific genes deleted from their genome (knockouts) for research or commercial purposes;

7) To detect the presence or absence of infection in organisms and/or to determine the likely consequences of foreign organisms in biological entities (life forms);

8) To detect and explore the status of the host/parasite and infectious states, which may occur in nature as an adaptation for survival, as a benign or necessary process, or as a state of occult or latent infection. In some instances these infectious states may act as a sensitising event that predisposes the host to the subsequent development of diseases such as autoimmunity or malignancy,

9) To allow the genetic fingerprinting of organisms including infectious agents known to be highly prone to genetic variations for example Influenza and Human Immunodeficiency viruses;

10) To conduct research into the function, regulation and importance of individual and interdependent genes and the regulation of specific processes and body functions in health and disease;

11) The ability to confirm the success or failure of processes that attempt to insert new nucleotide sequences into DNA/RNA for whatever reason, and conversely to determine the absence of such manipulation in whole animals or plant forms, or in products arising from such animals or plants, where they are specifically cultivated for human use or consumption as food or drugs or nutritional products;

12) The ability to monitor the status of cellular DNA/RNA following exposure to chemical, physical or other environments including extraterrestrial conditions that are know to produce random or specific damage to DNA (mutagenesis). At the present time this includes such known factors as ionising radiation, mutagenic chemicals, dietary contaminants or certain infectious agents but may be extended by discoveries in the future;

13) The ability to monitor the status, of cellular DNA/RNA following cloning life forms. Such processes may demand confirmation that the cloned organisms possess intact or undamaged DNA sequences or homopduplexes;

14) The ability to confirm that native DNA/RNA is not damaged, broken, looped substituted or deleted in organisms and exist in as form that does not differ form its naturally occurring state, in the cell nucleus or other forms when it exists in the cell cytosol.

Nucleic acid molecules are polymers of nucleotides in which the 3′ position of one nucleotide sugar is linked to the 5′ position of the next by a phosphodiester bridge. Nucleotide molecules contain the bases thymine (T), cytosine (C), guanine (G) and adenine (A) in the case of DNA and C, G, A and uracil (U) in the case of RNA. Double stranded nucleic acid molecules result from the formation of a duplex of complementary base pairing where C and G bind together and A and T bind together (DNA) or U and A bind together (RNA). DNA can also be produced using abnormal and derived bases such as uracil.

The sequence of bases determines the sequence of amino acids of the proteins encoded by the nucleic acid molecules. Alterations in the nucleotides result in variations in the amino acid sequence which may in turn affect 3 dimensional structure and protein function. Abnormalities in DNA nucleotide sequences may affect both the production and the function of dependent proteins ranging from lack of production of the protein, to truncations of the amino acid sequence or single or multiple amino acid substitutions, deletions or insertions.

The ability to detect mutations and small insertions or deletions in nucleic acid molecules is becoming increasingly important in the field of genetic testing of disease conditions or the propensity for the development of disease conditions. Existing methods, such as single strand conformation polymorphism (SSCP) and the chemical cleavage of mismatch (CCM) method are expensive and time consuming, particularly as they include a separation step to separate single strands or fragments which are not always easily resolved. The CCM method also employs a cleaving agent, usually piperidine which is toxic, thus incorporating an additional step which presents further safety and handling problems in the protocol.

Thus, there remains a need for further protocols for detecting mutations in nucleic acid sequences or detecting differences between nucleic acid duplexes which avoid the use of cleaving agents and the time consuming step of separation of single strands or fragments, or provides greater resolution or discrimination (ie enhanced separation) in methods relying on a separation step.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference to any prior art in this specification is not, and should not be taken as, an acknowledgment or any form of suggestion that that prior art forms part of the common general knowledge in Australia

The subject specification contains nucleotide sequence information prepared using the programme PatentIn Version 3.1 presented herein after the references. Each nucleotide sequence is identified in the sequence listing by the numeric indicator <210> followed by the sequence identifier (e.g. <210>1, <210>2 etc). The length, type of sequence (DNA) and source for each nucleotide sequence are indicated by information provided in the numeric indicator fields <211>, <212> and <213>, respectively. Nucleotide sequences referred to in the specification are defined by the information provided in the numeric indicator field <400> followed by the sequence identifier (eg <400>1, <400>2 etc). Each sequence in the listed sequence information is read from left to right in the 5′ to 3′ direction.

It has now been found that base pairing differences between nucleic acid molecules eg. a mismatched or unmatched nucleotide base in a nucleic acid heteroduplex, as compared with a control nucleic acid duplex (eg homoduplex), can be detected without a single strand or fragment separation step, based on determining the presence (consumption or formation) of starting agents and/or reaction products as a result of treating a nucleic acid duplex with an oxidizing agent. The invention allows for detection of a variation or mutation or a substituted base in a test nucleic acid molecule (first nucleic acid molecule) by hybridizing it with a control nucleic acid molecule (second nucleic acid molecule) to provide a test nucleic acid duplex (heteroduplex). A variation or mutation or substituted base in the test nucleic acid molecule will then be apparent as a base pairing difference (mismatched or unmatched base) in the test nucleic acid duplex. Thus, the present invention relates to a method for detecting a base pairing difference between a first nucleic acid molecule and a second nucleic acid molecule, comprising the steps of treating a duplex formed from said first and second nucleic acid molecules with an amount of an oxidizing agent sufficient to oxidize a mismatched or unmatched base in the nucleic acid duplex and then monitoring the formation, or the rate of formation, of one or more reaction products and/or consumption, or the rate of consumption, of one or more starting agents. By separately treating a control duplex under the same conditions and comparing the oxidation reaction with that of the test duplex, a base pairing difference between the first and second nucleic acid molecule can be identified.

The invention also provides a method for detecting a difference between two different nucleic acid duplexes, even where there are no mismatched or unmatched nucleotide bases present in the duplexes, by determining the extent of oxidation of each duplex when treated with an oxidizing agent, and then determining if there is a difference between the extent of oxidation for the two duplexes.

According to the invention, there is provided a method for detecting a base pairing difference between a first nucleic acid molecule and a second nucleic acid molecule in a test nucleic acid duplex comprising the steps of:

(i) treating the test nucleic acid duplex with an effective amount of an oxidizing agent for a time and under conditions sufficient to oxidize a mismatched or unmatched base in the test nucleic acid duplex;

(ii) monitoring the formation, or rate thereof, of one or more reaction products; and/or the consumption, or rate thereof, of one or more starting agents; and

(iii) determining if there is a difference in the formation, or rate thereof, of one or more reaction products and/or the consumption, or rate thereof, of one or more starting agents between the test nucleic acid duplex and that of a control nucleic acid duplex which has separately been subjected to the same conditions of steps (i) and (ii);

wherein a difference in the formation, or rate thereof, of one or more reaction products and/or the consumption, or rate thereof, of one or more starting agents between the test and control nucleic acid duplexes is indicative of a base pairing difference between said first and second nucleic acid molecules.

The method of the invention thereby indirectly allows for the detection of the presence of a variation or modification, including a substituted base, in a nucleic acid molecule.

In an embodiment of the invention, the first nucleic acid molecule and second nucleic acid molecule are nucleotide sequences.

In another embodiment of the invention, the base pairing difference is the result of a point mutation, insertion or deletion in a nucleic acid molecule (ie a single base mismatch or unmatched base).

In another embodiment of the invention, the mismatched or unmatched base in the test nucleic acid duplex is thymine (DNA), cytosine (DNA or RNA) or uracil (RNA).

In another aspect, the present invention provides a method for detecting a difference between a first nucleic acid duplex and a second nucleic acid duplex, comprising the steps of:

(i) separately treating each duplex with an oxidizing effective amount of an oxidizing agent for a time and under conditions sufficient to at least partially oxidize at least one duplex;

(ii) monitoring the formation, or rate thereof, of one or more reaction products and/or the consumption, or rate thereof of one or more starting agents for each duplex; and

(iii) determining if there is a difference in the formation, or rate thereof, of one or more reaction products and/or the consumption, or rate thereof, of one or more starting agents between the first and second nucleic acid duplexes,

wherein a difference in the formation, or rate thereof, of one or more reaction products and/or the consumption, or rate thereof, of one or more starting agents is indicative of a difference between the first and second nucleic acid duplexes.

In further preferred embodiments of the invention, the oxidizing agent is potassium permanganate (KMnO₄).

In other preferred embodiments of the invention, the oxidation reaction is monitored by UV visible spectroscopy, or visual detection, including by the naked eye in a suitable device.

### BRIEF DESCRIPTION OF THE DRAWINGS

FIG. 1 depicts a comparative study of permanganate oxidation reactions with the 11 base pair homoduplex DNA 4 and the heteroduplex DNA 5 at 25° C. and 50° C. Series 1: duplex 4 (25° C.); series 2: duplex 5 (25° C.); series 3: duplex 4 (50° C.)and series 4: duplex 5 (50° C.). The duplexes (20 nmol each) were reacted with with KMnO₄ (100 nmol) in 3M TEAC solution.

FIGS. 2a and 2b depict successive scans and determination of isosbestic points for the oxidation reactions with the duplex 5 at 25° C. and the duplex 4 at 25° C. respectively. The duplexes 4 and 5 (20 nmol each) were reacted with KMnO₄ (100 nmol) in 3M TEAC solution.

FIG. 3 depicts UV-Visible spectra of the permanganate oxidation reactions with the test 547 base pair heteroduplex DNA (curve 1), homoduplex DNA (curve 2) and the control (no DNA, curve 3). The test duplexes (mouse β-globin promoter, 12.4 μg) were reacted with KMnO₄ (0.2 nmol) in 3M TEAC solution at 25° C.

FIG. 4 depicts the oxidation spectra of duplexes 12 (upper curve) and 13 (lower curve) with initial oxidation temperatures of 53° and 59° C.

FIG. 5 depicts the oxidation spectra of duplexes 14 (upper curve) and 15 (lower curve) with initial oxidation temperatures of 51° and 55° C.

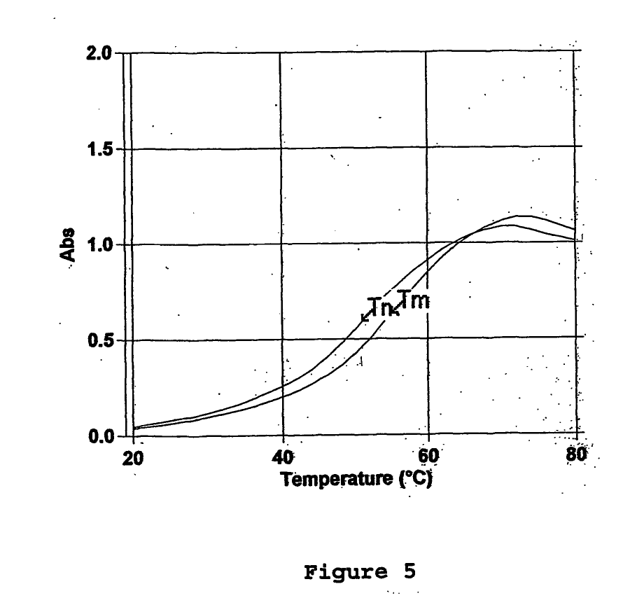

FIG. 6 depicts the sequence for 547 basepair mouse β-globin promoter homoduplex (wildtype)².

FIG. 7 depicts the sequence for 547 basepair mouse β-globin promoter homoduplex (mutant) for duplex samples 16 wherein the 5′T-3′A nucleotide pair at position 107 of the wildtype homoduplex (reading the duplex sequence from left to right) is replaced by a 5′C-3′G match (as identified by the surrounding box).

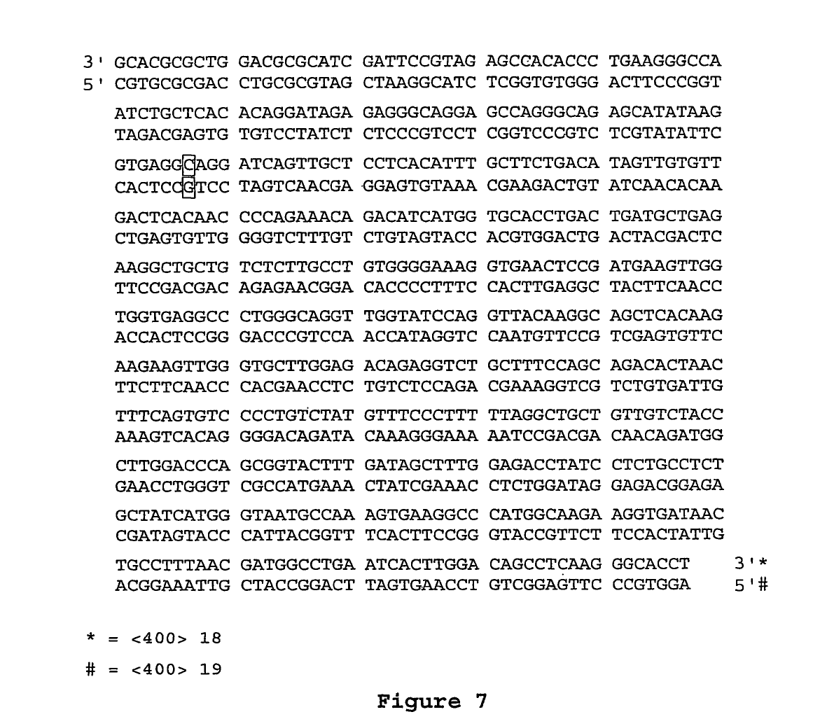

FIG. 8 depicts the sequence for 547 basepair mouse β-globin promoter heteroduplex (C-A mismatch) for duplex samples 16 wherein the 5′T-3′A nucleotide pair at position 107 of the wildtype homoduplex (reading the duplex sequence from left to right) is replaced by a 5′C-3′A mismatch (as identified by the surrounding box).

FIG. 9 depicts the sequence for 547 basepair mouse β-globin promoter heteroduplex (T-G mismatch) for duplex samples 16 wherein the 5′T-3′A nucleotide pair at position 107 of the wildtype homoduplex (reading the duplex sequence from left to right) is replaced by a 5′T-3′G mismatch (as identified by the surrounding box).

FIG. 10 depicts the sequence for 547 basepair mouse β-globin promoter homoduplex (mutant) for duplex samples 17 wherein the 5′C-3′G nucleotide pair at position 82 of the wildtype homoduplex (reading the duplex sequence from left to right) is replaced by a 5′A-3′T match (as identified by the surrounding box).

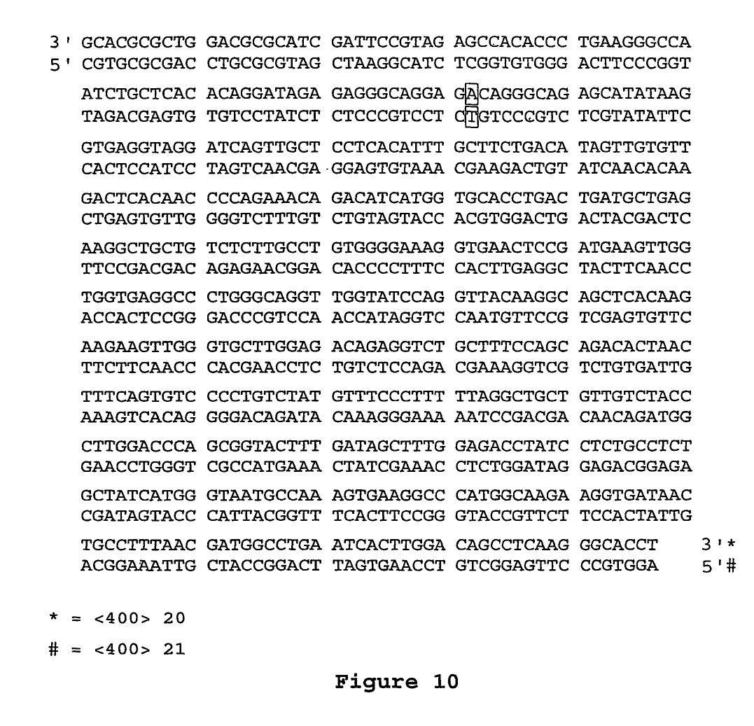

FIG. 11 depicts the sequence for 547 basepair mouse β-globin promoter heteroduplex (C-T mismatch) for duplex samples 17 wherein the 5′C-3′G nucleotide pair at position 82 of the wildtype homoduplex (reading the duplex sequence from left to right) is replaced by a 5′C-3′T mismatch (as identified by the surrounding box).

FIG. 12 depicts the sequence for 547 basepair mouse β-globin promoter heteroduplex (A-G mismatch) for duplex samples 17 wherein the 5′C-3′G nucleotide pair at position 82 of the wildtype homoduplex (reading the duplex sequence from left to right) is replaced by a 5′A-3′G mismatch (as identified by the surrounding box).

FIG. 13 depicts the sequence for 547 basepair mouse β-globin promoter homoduplex (mutant) for duplex samples 18 wherein the 5′C-3′G nucleotide pair at position 83 of the wildtype homoduplex (reading the duplex sequence from left to right) is replaced by a 5′G-3′C match (as identified by the surrounding box).

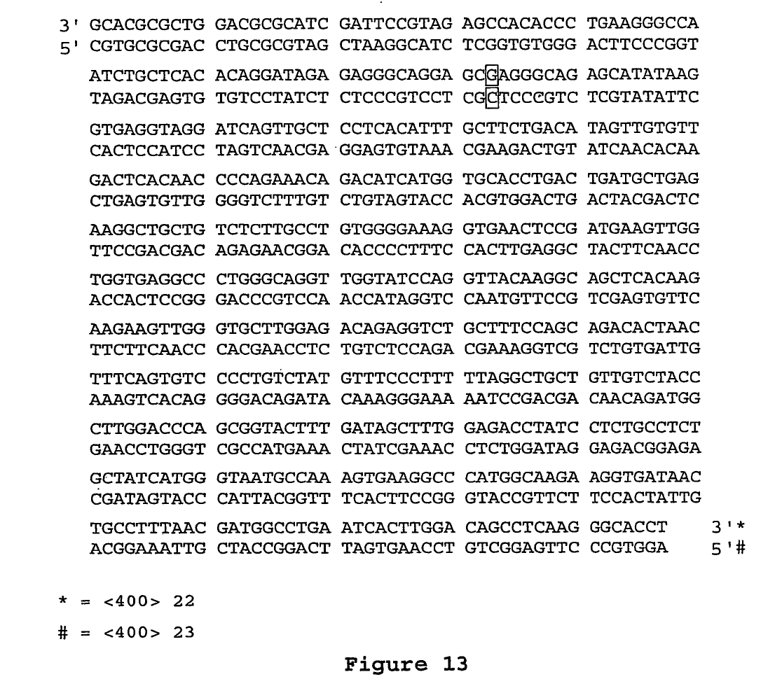

FIG. 14 depicts the sequence for 547 basepair mouse β-globin promoter heteroduplex (G-G mismatch) for duplex samples 18 wherein the 5′C-3′G nucleotide pair at position 83 of the wildtype homoduplex (reading the duplex sequence from left to right) is replaced by a 5′G-3′G mismatch (as identified by the surrounding box).

FIG. 15 depicts the sequence for 547 basepair mouse β-globin promoter heteroduplex (C-C mismatch) for duplex samples 18 wherein the 5′C-3′G nucleotide pair at position 83 of the wildtype homoduplex (reading the duplex sequence from left to right) is replaced by a 5′C-3′C mismatch (as identified by the surrounding box).

FIG. 16 depicts the sequence for 547 basepair mouse β-globin promoter homoduplex (mutant) for duplex samples 19 wherein the 5′T-3′A nucleotide pair at position 123 of the wildtype homoduplex (reading the duplex sequence from left to right) is replaced by a 5′A-3′T match (as identified by the surrounding box).

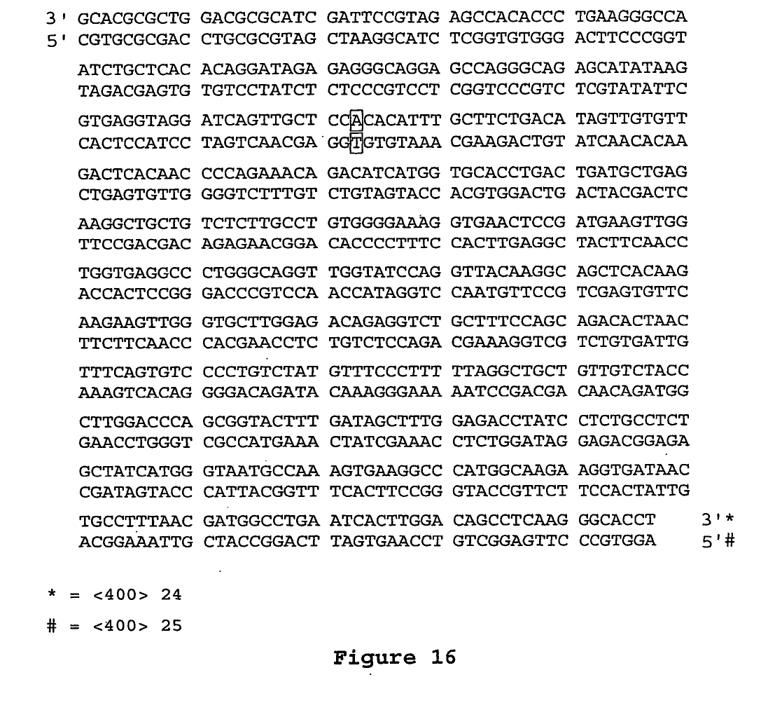

FIG. 17 depicts the sequence for 547 basepair mouse β-globin promoter heteroduplex (A-A mismatch) for duplex samples 19 wherein the 5′T-3′A nucleotide pair at position 123 of the wildtype homoduplex (reading the duplex sequence from left to right) is replaced by a 5′A-3′A mismatch (as identified by the surrounding box).

FIG. 18 depicts the sequence for 547 basepair mouse β-globin promoter heteroduplex (T-T mismatch) for duplex samples 19 wherein the 5′T-3′A nucleotide pair at position 123 of the wildtype homoduplex (reading the duplex sequence from left to right) is replaced by a 5′T-3′T mismatch (as identified by the surrounding box).

FIG. 19 depicts the oxidation analysis spectra of 547 base pair DNA (sample 16) containing T-G/A-C mismatches: heteroduplex (left), homoduplex-wildtype (middle) and homoduplex-mutant (right). Absorbance was measured at 420 nm.

FIG. 20 depicts the oxidation analysis spectra of 547 base pair DNA (sample 17) containing T-C/A-G mismatches: heteroduplex (left), homoduplex-wildtype (middle) and homoduplex-mutant (right). Absorbance was measured at 420 nm.

FIG. 21 depicts the oxidation analysis spectra of 547 bp DNA (sample 18) containing C-C/GG mismatches: heteroduplex (left), homoduplex-wildtype (middle) and homoduplex-mutant (right). Absorbance was measured at 420 nm.

FIG. 22 depicts the oxidation analysis spectra of 547 bp DNA (sample 19) containing T-T/AA mismatches: heteroduplex (left), homoduplex-wildtype (middle) and homoduplex-mutant (right). Absorbance was measured at 420 nm.

### DETAILED DESCRIPTION OF THE INVENTION

As used herein, the terms “oxidation”, “reaction” and “modification” (and variants thereof) can be used interchangeably.

As described above, complementary base pairing occurs in a double stranded nucleic acid duplex consisting of a single stranded nucleic acid molecule hybridized together with its complementary strand when G and C bases bind together and A and T bases bind together (or U and A bind together). A base not bound to its complementary base, but paired to another base instead, is referred to as a mismatched base and the pair referred to as a mismatched base pair, for example CC, CT, CA, TG, TT, AA, AG, GG, UG, UU or UC. Where a base is not bound to another base on the second strand, this is referred to as an unmatched base. The base pairing difference between the first nucleic acid molecule and the second nucleic acid molecule is to be understood to refer to a non-complementary base pairing, or non-pairing of a base ie a mismatched or unmatched base.

A control nucleic acid duplex is a duplex about which base pairing information is known, for example a fully complementary paired duplex (referred to as a homoduplex). However, in some circumstances, where appropriate, a control nucleic acid duplex may contain one or more mismatched or unmatched bases, provided the base pairing information is known. A homoduplex refers to a duplex where all the bases of the first and second nucleic acid molecules are paired in a complementary fashion and is preferably used as a control nucleic acid duplex,

A heteroduplex refers to a duplex wherein there is one or more mismatched or unmatched bases, and may also be referred to as a test nucleic acid duplex. A base pairing difference between the first and second nucleotide molecules may be the result of either modification, removed base (ie abasic site), substitution, addition or deletion of a single nucleotide or more than one nucleotide in a nucleic acid molecule (ie the test nucleic acid molecule). Such variations in a nucleotide sequence will result in base pair mismatches, or unbound (unmatched) base or bases when the first (test) and second (control) nucleotide sequences are hybridized to form a duplex. The method of the present invention is especially useful in that it provides for the detection of differences arising from a single base change in a nucleic acid molecule (referred to as a mutation or variation) giving rise to, on hybridization, a single base pair mismatch or unbound (unmatched) base. Thus, the present invention allows for the detection of one or more base mutations or variations in a nucleic acid molecule by hybridizing it with a control nucleic acid molecule (being a nucleic acid molecule which is complementary to the test nucleic acid molecule without the variations or mutations) and detecting for a base pairing difference (mismatched or unmatched base) within the test nucleic acid duplex.

The invention can also be used to detect a difference between two nucleic acid such as DNA homoduplexes from different species. A difference between two nucleic acid duplexes refers to two nucleic acid duplexes which are non-identical in terms of their nucleotide sequences and/or base pairing. Not only can differences between nucleic acid duplexes arise from a mismatched or unmatched base as described herein, but also from a difference in one or more matched nucleotide pairs (such as the homo-wildtype and homo-mutant duplexes of Example 8, or simply a difference arising from nucleic acid duplexes obtained from entirely different sources (see for example, Example 5). Thus, the invention also relates to a method for detecting a difference between a first nucleic acid duplex and a second nucleic acid duplex comprising the steps of separately treating said first and second nucleic acid duplexes with an oxidizing effective amount of an oxidizing agent for a time and under conditions sufficient to at least partially oxidize at least one duplex and determining the relative extent of oxidation of each duplex after a predetermined time, wherein a different extent of oxidation (as determined, for example by isosbestic point) of each duplex is indicative of a difference between the first and second nucleic acid duplexes.

As used herein, the term “nucleotide” is taken to refer to the monomeric unit which comprises a phosphate group, a sugar moiety and a nitrogenous base. Preferred sugar moieties are the pentose sugars, such as ribose and deoxyribose, however, hexose sugars are also to be considered within the scope of the term “sugar group”. The nitrogenous base is taken to refer to any nitrogen containing moiety which can act in pairing or mispairing in a nucleic acid duplex and as a proton acceptor. Preferred nitrogenous bases are cyclic, comprising preferably of one, or more rings (e.g. mono- or bi-cyclic) and contain at least one nitrogen atom. Preferred nitrogenous bases include the pyrimidine bases such as uracil, thymine and cytosine, and the purine bases such as adenine and guanine or simple derivatives thereof (ie substituted bases) such as deazapurines and inosine.

As used herein, the term “nucleic acid molecule” is taken to refer to a “single stranded” molecule comprising at least two nucleotides, ie, a nucleic acid duplex has at least 2 pairs of nucleotides. Thus the methods of the invention may be applied to nucleic acid molecules (or duplexes) having from 2 nucleotides (or 2 pairs of nucleotides) to whole genomes.

As used herein, a “nucleotide sequence” is taken to refer to a linear sequence of nucleotides selected from:

| | |
|---|---|
| 2′-Deoxycytidine 5′-phosphate; | Cytidine 5′-phosphate; |
| 2′-Deoxyadenosine 5′-phosphate; | Adenosine 5′-phosphate; |
| 2′-Deoxyguanosine 5′-phosphate; | Guanosine 5′-phosphate; |
| 2′-Deoxythymidine 5′-phosphate; | Uridine 5′-phosphate |

or simple derivatives thereof such as deazapurines and inosine.

Thus, in a preferred embodiment of the present invention, the first and second nucleic acid molecules are nucleotide sequences.

The nucleic acid duplexes may be obtained commercially, synthetically or obtained from nucleic acid duplexes by melting and re-annealing and may be derived from purified genomic DNA or RNA, or PCR products or from DNA/RNA that is present is-situ in cells or tissues that have been affixed to some form of solid matrix suitable for examination by transmitted or reflected radiation including light microscopy, electron microscopy, confocal microscopy or similar technology. The methods of the invention may be used to detect a difference between two nucleic acid molecules by hybridizing a first “test” nucleic acid molecule, which may contain one or more mutations, with a second “control” nucleic acid molecule which contains no mutations to form a test nucleic acid duplex (heteroduplex). Two test nucleic acid duplexes can be prepared by mixing, melting and reannealing a control nucleic acid duplex and a test-mutant nucleic acid duplex, being fully complementary paired and containing one or more sequence variations compared to the control nucleic acid duplex. (see for example, Example 8). This allows for the preparation of complementary mismatched base pairs or unmatched bases. This hybridization can be performed using methods known in the art and may occur during the PCR process when the natural DNA or RNA is amplified. Mismatched base pairs and/or unmatched bases may then be detected. Thus, the presence of genetic variations or mutations in a test nucleic acid molecule (ie the test strand) can be detected. One suitable type of duplex is locked DNA which may allow reaction at higher temperatures and thus reduce oxidation due to melting.

It has been found that mismatched or unmatched nucleotide bases in a duplex may be selectively more reactive towards an oxidizing agent compared to a matched or paired nucleotide base. Suitable oxidizing agents for use in the present invention may include KMnO₄, OsO₄, chromic acid, ozone gas, peroxides (eg H₂O₂) and perbenzoic acids (eg m-chloroperbenzoic acid and derivatives thereof). A particularly suitable oxidizing agent is KMnO₄.

An oxidizing effective amount of an oxidizing agent is an amount sufficient to modify (oxidize) an unmatched or mismatched base in a nucleic acid duplex to the extent that the consumption of one or more starting agents or the formation of one or more products can be detected.

A starting agent is an agent which is used in the oxidizing reaction, such as the oxidizing agent or the nucleic acid duplex under consideration. A reaction product is a product formed as a result of the oxidation of an mismatched or unmatched base in the duplex, such as the oxidized nucleic acid duplex or the reduced form of the oxidizing agent.

Nucleic acid molecules can be either end-labelled or unlabelled. By use of a labelled (either end labelled or internally labelled) DNA or RNA as appropriate, it may be possible to obtain information about the location of mutations. Any convenient label may be used, including, eg. radioactive labels, fluorescent labels and enzyme labels in a manner well known to those skilled in the art. Suitable labels include: ³²P, ³³P, ¹⁴C, FAM, TET, TAMRA, FLUORESCEIN, and JOE.

The oxidization of the nucleic acid duplex can be performed with all the starting agents in solution or by immobilizing the duplex onto a solid support matrix. In certain embodiments of the invention, immobilizing the duplex onto a solid support may be advantageous as it allows for the ready separation of the duplex from reaction solution and may thus simplify the detection of starting agents and/or reaction products. Suitable solid supports may be made of an appropriate polymeric material, be silicon derived (eg silica/glass) or paper. Supports may be in the form of pins, wells, plates or beads and may have a magnetic component or may be fully or partially coated with streptavidin so as to allow for attachment of a biotinylated duplex. In immobilizing the duplex to the solid support, this may be done by attaching the duplex to the support, or alternatively, attaching the first nucleic acid molecule to the support and then hybridizing the second nucleic acid molecule to it to form the attached duplex.

Determination of the presence of starting agents and/or reaction products, ie monitoring the extent or rate of formation of one or more reaction products and/or the extent or rate of consumption of one or more starting agents, can be carried out by any suitable means which may include spectroscopy (eg UV visible, NMR, mass spectrometry), microscopy, chromatography (eg HPLC, GC), titration, colorimetry, inorganic assay for the detection of oxidizing agent or reduced form thereof (eg MnO₂) and electrochemical detection wherein a change in electrical current is indicative of a redox reaction. The oxidized nucleic acid duplex may also be detected by coupling the oxidized mismatched or unmatched base to another organic molecule (eg an aldehyde) or another redox reagent system eg a redox stain, and detecting the formation of the resulting coupled product by a suitable means, for example as described above. In certain embodiments of the invention it may be useful to determine the presence of the oxidizing agent and/or the reduced form of the oxidizing agent.

The oxidizing reaction for detecting a difference between a first nucleic acid molecule and a second nucleic acid molecule may be carried out in the range of about 0° C. to the melting point of the duplex, such as about 10-50° C. Preferably the oxidation is performed in the temperature range of about 20-40° C., more preferably at about 25° C. or 37° C. The oxidation reaction for identifying differences between different nucleic acid duplexes can be carried out at the temperatures as described above but can also be carried out above the melting point of the duplex by comparing rates of oxidation due to differing numbers of reactive bases (eg T or C bases) in each duplex.

The rate of modification of the mismatched or unmatched base depends on the nature of the base itself Certain oxidizing reagents (eg KMnO₄, OsO₄) are more selective towards unmatched or mismatched thymine and uracil while the rate of the reaction with cytosine is slower. Rates of reaction are generally lower still where the mismatched or unmatched base is guanine or adenine. Thus, preferably, the mismatched or unmatched base to be modified is thymine, uracil or cytosine. Preferably where there are two complementary pairs of mismatched or unmatched bases, these will include thymine (or uracil) and cytosine as this may allow for the detection of all mutations and give each mutation two chances of detection. Neighbouring matched bases may also be reactive, especially as the duplex starts to melt.

The time taken for the oxidation may be dependent on the reaction temperature and the nature of the mismatched or unmatched base to be modified. Preferably the time is in the range of about 1 minute to about 10 hours, eg. from about 5 minutes to about 3-4 hours. Preferably the modification is performed for about 10 minutes to about 1 hour, eg. about 30 minutes.

The modification is suitably carried out in aqueous solution or a mixture of aqueous and non-aqueous solvents and may, where appropriate, be performed under acidic, neutral or basic conditions, and may optionally be performed in the presence of other agents such as a buffer, eg citrate or phosphate buffer. In one embodiment, the modification can be carried out in the presence of an amino base or salt thereof. Suitable amino bases may include alkyl amines (mono- and di-) and suitable salts thereof include sulfates, nitrates and halide salts, for example chloride. Examples of bases include tetraethylamine, tetramethylamine diisopropylamine, tetraethylene diamine hydrazine and pyridine. Examples of preferred ammonium salts include tetraethylammonium chloride (TEAC) and tetramethylammonium chloride (TMAC). The base (or salt) solution may be of a concentration of between about 0 to about 6 M, preferably about 2-4 M, particularly about 3M.

In one preferred embodiment of the invention, the oxidizing agent is KMnO₄. Permanganate oxidation (modification) of a free nucleotide base (such as thymine) results in the formation of an unstable intermediate cyclic permanganate diester which decomposes under basic conditions to release the diol and soluble MnO₂ (Scheme 1).

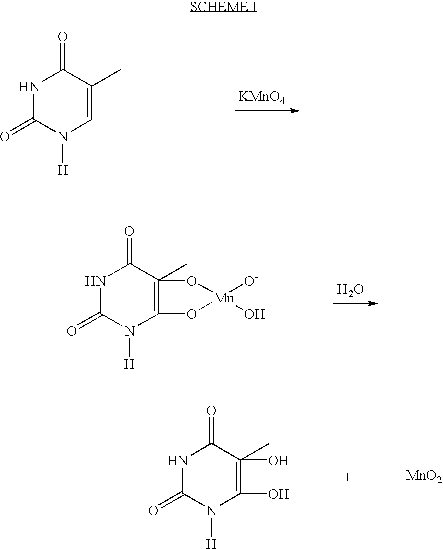

MnO₂ absorbs strongly at 420 nm whereas MnO₄⁻ is almost transparent at this wavelength. However, MnO₄⁻ exhibits strong absorption at 525 nm. Thus, conveniently, the oxidation reaction can be monitored by UV spectroscopy at a wavelength in the range of about 400-440 nm, more preferably in the range of 410-430 nm, such as about 420 nm for the formation of MnO₂ and/or in the range of about 505-545 nm, more preferably in the range of 515-535 nm, such as about 525 nm for the consumption of KMnO₄.

Preferably the KMnO₄ is used in a molar excess per mismatched or unmatched base, for example at least about 3 molar excess, more preferably about 5 molar excess, if the formation of MnO₂ is being detected. If the consumption of KMnO₄ (MnO₄⁻) is being monitored, KMnO₄ is preferably used in an approximately equimolar amount per mismatched or unmatched base. Oxidation using KMnO₄ may be carried out in the presence of TEAC or TMAC, or without. In one embodiment of the invention, the oxidation may be carried out in a solution of TEAC or TMAC.

In a preferred embodiment, a mismatched or unmatched T base, U base or C base is modified by KMnO₄.

Since the two manganese species both give strong absorption in the visible region, determination of the presence of MnO₄⁻ or MnO₂ can also be carried out by simple visual analysis, for example, MnO₄⁻ exhibits a pink colour in TEAC while MnO₂ exhibits a yellow colour in TEAC.

The presence of a mismatched or unmatched base can also be determined by comparison of the respective isosbestic points for a heteroduplex ie. the test nucleic acid duplex containing the mismatched or unmatched bases and its corresponding homoduplex ie. the control nucleic acid duplex which contains no mismatched or unmatched bases. The isosbestic point in an absorption spectrum of two substances (eg. MnO₂ and MnO₄⁻) in equilibrium with each other is the wavelength at which the two substances have the same molar extinction coefficients. By sequential scanning over a suitable time interval in the UV-visible region, the isosbestic point for the modification of a nucleic acid sample can be determined. Matched nucleotide bases in a homoduplex react with an oxidizing agent more slowly than a mismatched or unmatched base in a heteroduplex. Thus after a predetermined interval, the isosbestic point for a heteroduplex would be expected to be different that that of a homoduplex. The isosbestic point can be used in combination with the rate of change of absorbance to obtain more accurate determinations.

A relative comparison of the isosbestic point for two nucleic acid duplexes can also be used to detect a difference between two nucleic acid duplexes, eg nucleic acid duplexes derived from different sources, such as DNA from different species even if there are no mismatched or unmatched bases in one or both of the duplexes as the rates of oxidation over a predetermined time interval would be expected to be different. Oxidative methods for detecting the difference between two nucleic acid duplexes can be performed as those described herein for detecting the difference between a first and second nucleic acid molecule.

The melting temperature of the heteroduplex is likely to be decreased by the presence of an oxidized base over the presence of a mismatched un-oxidized base and particularly over the homoduplex. Thus, in another aspect of the invention, the oxidation methods described herein can be used to enhance existing techniques ie separation techniques for deterring the presence of a modified nucleic acid duplex. Thus, in other embodiments of the invention, the formation of an oxidized heteroduplex and/or the consumption of the starting heteroduplex can be determined or detected by methods relying on melting temperature, for example by comparing the difference between the melting temperature of an oxidized heteroduplex and the starting heteroduplex or corresponding homoduplex. In such embodiments of the invention, detection of a mismatched or unmatched base by oxidation methods (such as using KMnO₄ as described above) can be used in conjunction with an increasing temperature gradient (such as about 2° C./minute). Thus the oxidation method is enhanced by the differential melting temperatures between a homoduplex and a heteroduplex containing the mismatch or unmatched base, wherein the heteroduplex has a lower initial melting temperature and therefore becomes more susceptible to oxidation by the oxidizing agent. The reacted mismatched and nearby freshly unmatched bases have the effect of further reducing the melting temperature of the heteroduplex, accentuating the difference in melting temperatures of the heteroduplex and homoduplex.

The melting temperature of DNA duplexes can be readily measured with modern technology by straight absorbence or by adding a double stranded specific dye (eg. Syber green I) to the oxidized heteroduplex and homoduplex and gradually increasing the temperature. As more and more single stranded DNA is produced the fluorescence is decreased which can be readily detected and the difference shown. Use of a single stand specific dye will also show the melting curve.

Suitable methods include Conformation Selective Gel Electrophoresis (CSGE), Denaturing Gradient Gel Electrophoresis (DGGE) or denaturing High Pressure Liquid Chromatography (dHPLC), wherein their discrimination is likely to be enhanced by the oxidative process.

Methods such as CSGE, dHPLC or DGGE rely on the discrepancy in melting temperature between a homoduplex and corresponding heteroduplex. However, in certain instances, this discrepancy in melting temperature may not be sufficient to be adequately resolved and indicate the presence of a mismatched or unmatched base. However, an oxidized heteroduplex, wherein a mismatched or unmatched base has been oxidized by an oxidizing agent, would be expected to melt at a lower temperature than that of the unoxidized heteroduplex, thus providing a greater difference in melting temperature compared to the homoduplex. This greater difference may aid in resolution, thus making “melting temperature” techniques more useful in identifying duplexes which contain a mismatched or unmatched base.

In the case of DGGE, it is expected that the physical event on which the method relies can be detected without separation of the oxidized heteroduplex, unoxidized heteroduplex and homoduplex. Thus in this method a sudden denaturing (opening) of the duplex occurs during a slowly increasing temperature or denaturing concentration during electrophoretic separation. This opening will happen sooner in the heteroduplex and is expected to occur even earlier after oxidation of the heteroduplex. The homoduplex opens later and moves further. Thus, the heteroduplex, and hence the mutation, can be detected. If the denaturant (eg temperature, or chemical denaturant) is slowly increased in the presence of an oxidizing agent it would be expected that a sudden increase in consumption of oxidizing agent (or formation of product) when the duplex opens, would be detected. This would occur earlier for a heteroduplex than the corresponding homoduplex.

Another method of detecting the mismatched or unmatched base is by use of allele specific oligonucleotide hybridization which can be carried out on chips, beads, pins, wells etc or in liquid phase. Thus, the temperature at which the oxidized heteroduplex will melt and hybridize with another piece of DNA (eg a probe) will be lower than that for the corresponding unoxidized heteroduplex, thereby potentially providing a greater differential hybridization signal, and allowing for easier detection of a mismatched or unmatched base.

Other separation methods which may be enhanced by the oxidative processes described herein include SSCP and sequencing, being methods known in the art. Agarose gels may be used to detect reaction products. The methods of the invention may be further used in conjunction with other reagents that react with mismatched or unmatched bases such as hydroxylamine or carbodiimide. Thus, such reagents may show enhanced reactivity with a mismatched or unmatched base after the mismatched base has been reacted with the oxidizing agent (eg KMnO₄). Alternatively, oxidation of the mismatched or unmatched base may be enhanced by firstly reacting the mismatched or unmatched base with the reagent. Conditions for reaction of hydroxylamine or carbodiimide with mismatched or unmatched bases are described in, for example, EP 329 311 and Novack et al, PNAS, 83, 586-60 respectively. Other reagents may include enzymes such as repair enzymes (eg mut Y, mut A, excision nucleases, s1 nuclease and resolvases).

In a further embodiment of the invention, a difference between two nucleic acid duplexes can be detected by carrying out the modification at a temperature just below the melting temperature of a heteroduplex. Thus, when both a heteroduplex and a homoduplex are each reacted with an oxidizing agent at a temperature just below the melting temperature of the heteroduplex, an oxidized heteroduplex so formed will melt thus exposing T & C bases (ie, now unmatched bases). This will result in a “burst” of oxidization activity for the heteroduplex which can be monitored by techniques described herein, eg by MnO₂ formation or KMnO₄ consumption.

In a further aspect of the invention, components and/or reagents for performing the present invention may be presented in a kit. The kit can be provided in compartmentalized form adapted for use in the present invention and may include one or more of: oxidizing agent, base (or salt thereof), test nucleic acid molecules or duplexes, buffers, spectroscopic cells and solid support phases, and may further be provided with instructions for performing the invention.

The method of the present invention is particularly useful for the screening of genetic material from mammalian cells, (eg. human; simian; livestock animals such as cows, goats, sheep, horses, pigs; laboratory test animals such as rats, mice, guinea pigs, rabbits; domestic companion animals such as dogs, cats; or captive wild animals), fish cells, reptile cells, bird cells, insect cells, fungi cells, bacterial cells or viral agents, parasitic agents, (eg. Plasmodium, Chlamydia, Rickettsia and protozoa) and plant cells including tobacco, ornamental trees, shrubs and flowering plants (eg. roses), trees, plants which product fruits and vegetables for human or animal consumption (eg. apples; pears; bananas; citrus fruit; stone fruit, including peaches, cherries, plums; potatoes; root vegetables; cabbage family etc) and agricultural crops such as oats, corn, barley, rye, cotton, sunflower, wheat, rice and legumes such as peas and soya, and laboratory test plants such as Aribidopsis Thalnianna.

The invention may also be particularly applicable to screen multiple samples in a high throughput fashion.

Some useful applications of the detection methods of the present invention are described below.

In all life forms currently known or envisaged, the ability to detect or predict the presence of genetic mutations and variations or damage to the integrity of the cellular DNA and RNA has applications in a variety of circumstances including but not limited to

Inherited Disease

The identification of inherited states where changes in the sequences of DNA in the chromosomes or genes may,

a) Confer desirable benefits such as enhanced immune, neuromuscular, cardiovascular intellectual or physical performance;

b) Confer disabilites due to reduced or absent performance of genentic mechanisms that control both the health of the organism and its response top changes in its environment and/or the occurrence of disease including;

I. Increased susceptibility to various disease including in some cases specific inherited diseases;

II. Disease or conditions which result from a failure to produce any/or sufficient amounts of a fully functional intracellular organelle, cellular process such as ingestion of, or secretion of intra or extracellular proteins other cell components especially protein and cell membrane receptor constructs on the cell membrane;

III. Failure of processes involved in normal cell fertilisation, development, organogenesis, cell function adaptation and repair and appropriately programmed cell death;

IV. Abnormal or inappropriate respons to external or internal environmental or dietary factors including infections, malignancies, inflammatory or degenerative disorders, nutritionsal diseases, auto-immune diseases, mental conditions including addictive, behavioural, psychotic, bi-polar and eating disorders;

V. Inappropriate activity of the immune system including allergy, autoimmunity and immunodeficiency.

The ability to accurately identify or predict inherited genetic mutations or differences offers a range of potential applications in the medical and diagnostic fields. It is particularly useful for examining DNA for known and unknown mutations in genes known or thought to cause disease in a high throughput mode. The method also allows for the detection of mutations in mRNA and there are situations where it may be of diagnostic use.

Identification of DNA & RNA Molecules

The ability to detect DNA and RNA molecules derived from different sources is important in such circumstances as the diagnosis of infections and the detection of genetically modified organisms. In principle, susceptibilities of DNA & RNA molecules towards permanganate ions are strongly dependent on their compositions of nucleotide bases and configurations. The described oxidation method can be applied for identification of DNA & RNA molecules, which are derived from different sources (plants, animals, human, viruses, etc.) and different strains or varieties of these. Example 5 describes a typical example for the comparative study between calf thymus DNA and mouse promoter DNA in terms of their isosbestic points.

Comparison of Related Virus Isolates

Viruses, typical examples of which include influenza and Human Immunodeficiency viruses, can mutate by changing the their nucleotide sequence rapidly in a short time. The usual method of comparison of these variant strains with a standard is sequencing and then comparison of sequences. Sequencing is tedious and subject to error, and the current method is capable of giving an indication of the difference between one virus and another as the number of mismatched/unmatched T and C bases are proportional to KMnO₄ reactivity.

Oncogenes or Tumour Suppressor Genes

The development of tumours including progressive malignancy is associated with mutations of genes involved in the regulation of cell growth and organogenesis. Which are processes controlled by naturally occurring local or systemic growth factors as well as genes controlling the normal life span of the cell and controlling programmed cell death. These genes are respectively called oncogenes and tumour suppressor genes and are important in the organism's continued normality and freedom from tumours. Many oncogenes or tumour suppressor genes which differ from normal by a single base have been characterised. Comparisons have been made by sequencing as for viruses. The method of the invention provides access to a rapid evaluation method for determining whether one oncogene has a mutation or difference relative to another. Such an ability could be valuable in the diagnosis, prognosis treatment and monitoring of patients with tumours.

Check of In Vitro Mutagenesis

Significant activity has been directed towards the alteration of specific bases in a gene to evaluate what effect this has on function. Essential to this is a need to determine (a) that the required base change has actually been effected, and (b) that other unwanted base changes have not been created. This is currently determined by sequencing and sequence comparison. The present invention offers a more convenient method for doing so.

Single Nucleotide Polymorphisms (SNP's) or Polymorphisms

Genes of living things contain variation in their sequences that are harmless as distinct from causing inherited disease or cancer. In the past, these changes were regarded as a nuisance to clinical geneticists and diagnosticians. In recent years they have moved to be of central importance in gene based medicine and have attracted much attention from the major pharmaceutical companies worldwide.

There are three major reasons for this:

1. Linkage mapping of disease. New genes that cause disease (or in the case of agricultural species which provide positive characteristics) are located and eventually identified by a process of linkage. In this process hundreds of “flags” or markers are identified along the genetic information and each in a family can be followed to see which one exactly is inherited with the disease (or trait) in question. This allows localization of the gene. The currently used markers need complex gel electrophoresis to assay them. SNP's offer a more simple assay, for example, on chips.

2. Identification of both more functional and less functional forms of genes occuring naturally as a result of SNPs may help in understanding and/or predicting pateint responses to certain therapeuitic drugs by identifying SNPs which alter during drug metabolism. Changes which reduce the function of a gene, for example, to 25%, without causing disease can cause administered drugs to give severe side effects. Identification of such SNP's could allow a preadministration genetic test to identify patients who will have a severe reaction so alternative treatment can be given.

3. The ability to identify SNPs in multiple genes in disorders such as asthma, diabetes, obesity autoimmune disease, cardiovascular disease could significantly improve the ability to diagnose and manage these conditions. Thus identification of multiple SNPs in the many common disorder genes (e.g. asthma, diabetes) could provide important understanding of these conditions. It is thought that genes with changes such as those described in 2, when combined with a number of such genes cause common disorders. Thus there is enormous activity to identify such genes by association studies.

Embodiments of the present method may offer the necessary high throughput mode to enhance these studies, especially with regard to cost, since no expensive separation step or cleaving agents are required.

Cardiovascular and Cerebrovascular Diseases

Cardiovascular and cerebrovascular diseases are a major cause of death and illness in the western world. In Australia alone, in 1996, of all deaths, 41.95% were due to heart disease or stroke compared to 26.93% due to cancer. Costs to the community are enormous not only in financial terms, but also in human terms.

Therapeutic Applications

The testing of the integrity of a DNA/RNA construct by the use of complementary DNA followed by testing using the oxidation mutation method can be adapted for use to enable quality control of nucleotide constructs for such therapeutic purposes as gene therapy, anti-sense oligonucleotide therapy, DNA vaccine production or other therapeutic modalities as may be developed using nucleotide constructs.

RNA Mutations

Mutations in RNA may also be detected. However, more complete information can be obtained by producing cDNA from the SS RNA of interest and testing this DNA with control DNA or directly hybridizing viral RNA with reference DNA.

Epidemiology

The ability to monitor changes or damage to genes or the total genome inpopulation groups could be of value as a means of ensuring enviromental quality and/or the absence of danger.

Industrial/Commercial Applications

The ability to synthesise DNA and RNA constructs allows the construction of nucleotide strings for pure industrial purposes. The ability to examine and identify scientific and industrial constructs by the use of complementary DNA followed by oxidation detection of unwanted variation, will enable such ‘designer’ nucleotides sequences as may be required in the future for industrial applications, to be confirmed as correct and in specification before use. Applications which can be envisaged include;

I. The use of single or multiple oligonucleotide sequences for identification and/or encryption

II. DNA based computing systems

III. To maintain the integrity of industrial products or commodities during transport to prevent substitution

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

The invention will now be described with reference to the following examples which are intended for the purpose of illustration only and are not to be construed as limiting the generality hereinbefore described.

### EXAMPLES

Chemicals and Spectrophotometer:

Chemicals, solvents and calf thymus DNA were purchased from Aldrich Chemical Company (Castle Hill, Australia). Oligonucleotides (HPLC grade) were purchased from Geneworks, South Australia Duplexes 4-15 were prepared by the preparation of the one-to-one mixture of single stranded nucleotides as per the previously described procedure.¹ Test samples of 547-base pair DNA (mouse β-globin promoter) were obtained and amplified as per the previously described conditions.² Calf Thymus DNA (CAS#[91080-16-19]) was purchased from Sigma-Aldrich Product No.D4522). Solutions were prepared in water. The silica beads (Ultraclean™ 15 purification kit) was purchased from MO BIO Laboratories, Inc, CA, USA. Oxidation reactions with potassium permanganate were carried out in a quartz 1.2 ml cuvette and the spectral data were obtained from Cintra-10 spectrophotometer (GBC Scientific Equipment Pty Ltd, Victoria, Australia) by recording the absorbance vs. time curves at pre-selected wavelengths and/or by repetitive scanning of the ultraviolet-visible region (200 to 800 nm).

### Example 1

In this study, two 11-residue oligonucleotide sequences, based on those in a previous study¹, were annealed as previously described to afford the homo and heteroduplex DNA. The resulting duplexes were subjected to a KMnO₄ solution in 3M TEAC solution at room temperature. The reaction was followed by measuring absorbance at 420 nm.

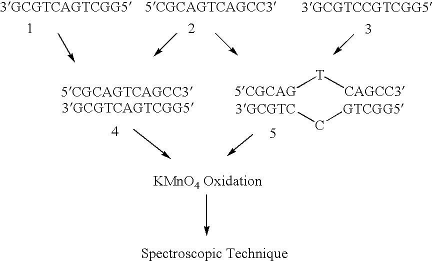

The model homo-duplex DNA 4 was prepared by preparation of one-to-one mixture of d(5′CGCAGTCAGCC3′) (2) and d(3′GCGTCAGTCGG5′) (1) and the hetero-duplex DNA 5 (which carries a T-C mismatch at the central position¹) was prepared by preparation of one-to-one mixture of d(5′CGCAGTCAGCC3′) (1) and d(3′GCGTCCGTCGG5′) (3).

For the oxidation reaction, 40 μl sample (20 nmol of 4 or 5 in distilled H₂O) was mixed with 10 μl KMnO₄ solution (100 nmol) in 0.95 ml of 3M TEAC solution. The reaction was followed by measuring absorbance at 420 nm and 525 nm over 30 min. Isosbestic points were determined by sequential scanning over 30 min at 25° C. and 50° C.

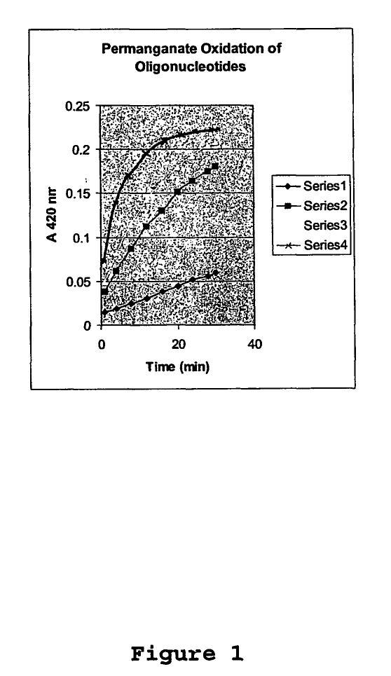

The results are depicted in FIGS. 1 (Series 1 (duplex 4 at 25° C.); Series 2 (duplex 5 at 25° C.); Series 3 (duplex 4 at 50° C.) and Series 4 (duplex 5 at 50° C.) and 2a (duplex 5 at 25° C.)-2b (duplex 4 at 25° C.)).

A further study was carried out at 25° C. varying the time of reaction (Table 1) and a preferred condition for these duplexes was found to be 25° C. at 30 min to achieve distinct discrepancy of MnO₂ levels obtained from two duplexes. Under these conditions, the rate of oxidation for the T-C mismatch contained in the heteroduplex 5 was almost two fold faster than the homoduplex 4.

TABLE 1
Percentage completion of the permanganate
oxidation reactions with two model
duplexes 4 and 5 at 25° C.:

| Time of reaction | % Reaction at 25° C. |
|---|---|
| min | Duplex 4/Duplex 5 |
| 5 | 9%/28% |
| 10 | 10%/41% |
| 15 | 17%/60% |
| 20 | 20%/70% |
| 25 | 24%/76% |
| 30 | 27%/83% |

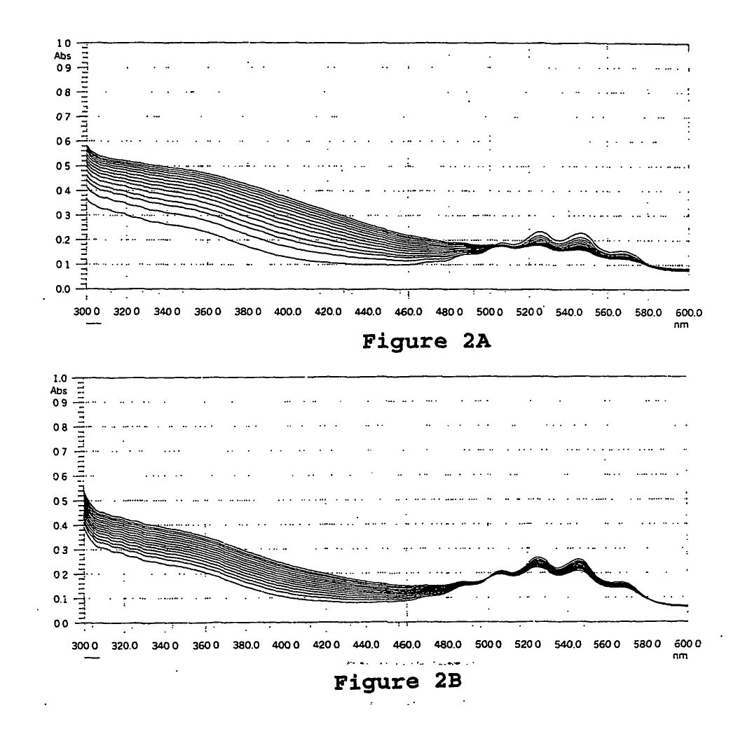

The uv-visible absorption spectra obtained by sequential scans from 200 to 800 nm of the oxidation reactions revealed significant differences in isosbestic points of the duplexes 4 and 5 at 25° C. (Table 2, FIGS. 2b and 2a).

TABLE 2
Isosbestic points of of DNA duplexes

| | Isosbestic point |
|---|---|
| DNA duplexes^{a} | at 25° C. |
| Duplex 4 | 500 nm |
| Duplex 5 | 504 nm |

^{a}Duplexes 4 (20 nmol) and 5 (20 nmol) were incubated with KMnO₄ (100 nmol). All reactions were carried out in 1 ml of 3M TEAC solution at 25° C.

### Example 2

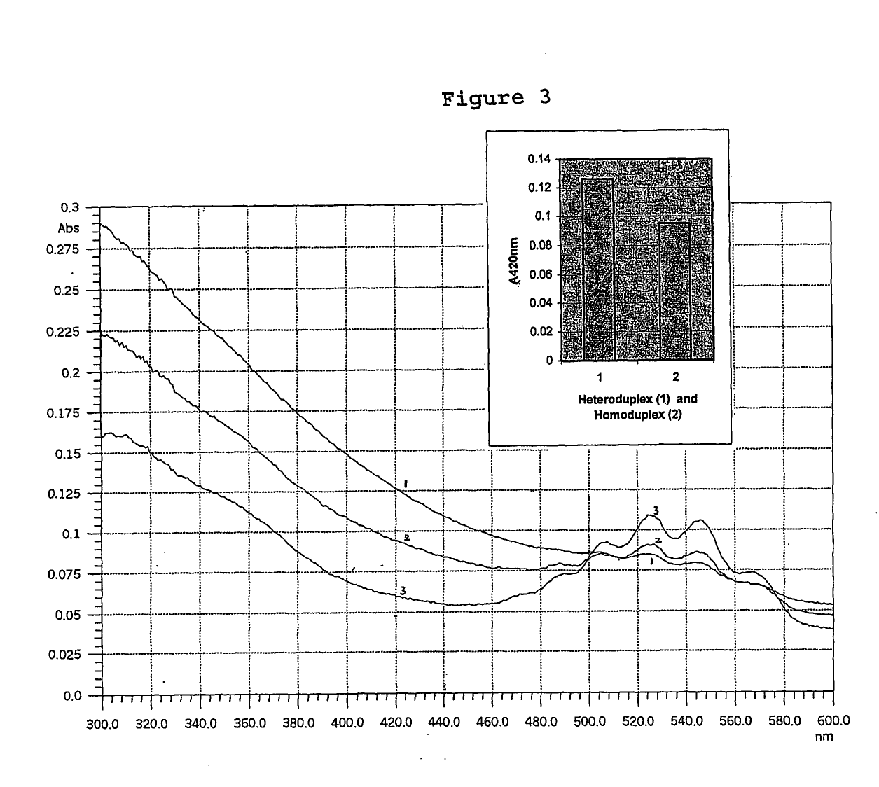

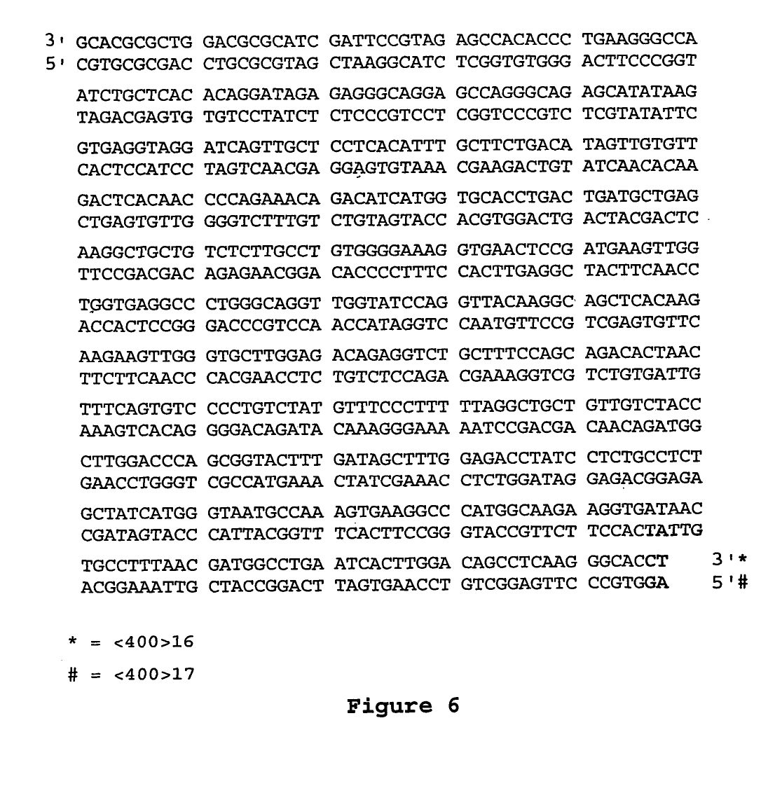

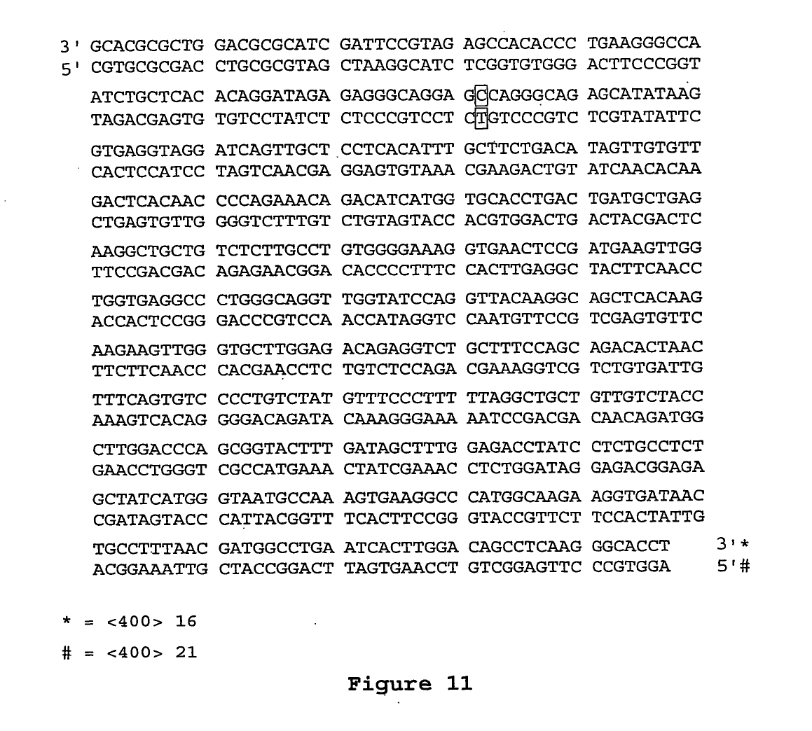

In this study the homo (wildtype, see FIG. 6) and heteroduplex (C-T mismatch, see FIG. 11) samples (547-basepair mouse promoter)² were immobilized on magnetic beads (in duplicate) and the resulting materials were treated with equal amounts of KMnO₄ at 25° C. for 15 min. The supernatant was immediately separated and measured at 420 nm and 525 nm. A difference in reactivity towards permanganate ions was observed between the two samples (FIG. 3). Immobilization of the DNA on a solid support (in these case beads) avoids spectral interference by the DNA.

The reactivity of KMnO₄ was studied with the model 547 bp wildtype and mutant DNA fragments which were amplified using fluorescently labeled primers (6-FAM for the 5′ primer, HEX for the 3′ primer). The sequence of the primers and PCR conditions was as previously reported¹. Formation of DNA homo and heteroduplexes were performed under previously reported conditions and subjected to the KMnO₄/TEAC oxidation reaction.² In general, the fluorescently labeled DNA was immobilized on silica beads. The resulting DNA bound beads (75-100 ng) were incubated with 20 μl of 1 mM KMnO₄/3M TEAC solution for 15 min at 25° C. After incubation time (15 min) the supernatant was immediately diluted to 1 ml of the 3M TEAC solution in a quartz cuvette. Comparative study for two test duplexes was followed by monitoring the production of MnO₂ and the disappearance of KMnO₄ which were quantitatively measured at 420 nm and 525 nm respectively. The results are depicted in FIG. 3 (heteroduplex: curve 1; homoduplex: curve 2; control (no DNA): curve 3).

### Example 3

Six model heteroduplexes DNA (Table 3) with four different types of mismatch (T-C, C-C, G-T and C-A) and their corresponding homoduplexes DNA were subjected to the permanganate oxidation test under conditions described above. Both the level of MnO₂ and isosbestic point were found to be altered in the heteroduplex in relation to the corresponding homoduplex.

TABLE 3

UV-visible Spectral Data for Permanganate Oxidation of the Model DNA Samples

| | | | | Level of |
|---|---|---|---|---|
| | | Mismatch or | Isosbestic MnO₂ | |
| # | Sequences | match (control) | point (nm) | at 30 min^{a} |
| 5 | 5′CGCAGTCAGCC3′ <400>1 | Mismatch T—C | 504 | 0.317 |
| | 3′GCGTCCGTCGG5′ <400>2 | | | |
| 4 | 5′CGCAGTCAGCC3′ <400>1 | Match T—A | 500 | 0.196 |
| | 3′GCGTCAGTCGG5′ <400>3 | | | |
| 6 | 5′CGCAGCCAGCC3′ <400>4 | Mismatch C—C | 503 | 0.373 |
| | 3′GCGTCCGTCGG5′ <400>2 | | | |
| 7 | 5′CGCAGCCAGCC3′ <400>4 | Match C—G | 499 | 0.234 |
| | 3′GCGTCGGTCGG5′ <400>5 | | | |
| 8 | 5′CGCAGGCAGCC3′ <400>6 | Mismatch G—T | 502 | 0.293 |
| | 3′GCGTCTGTCGG5′ <400>7 | | | |
| 9 | 5′CGCAGACAGCC3′ <400>8 | Match A—T | 510 | 0.201 |
| | 3′GCGTCTGTCGG5′ <400>7 | | | |
| 10 | 5′CGCAGCCAGCC3′ <400>4 | Mismatch C—A | 504 | 0.240 |
| | 3′GCGTCAGTCGG5′ <400>3 | | | |
| 4 | 5′CGCAGTCAGCC3′ <400>1 | Match T—A | 500 | 0.196 |
| | 3′GCGTCAGTCGG5′ <400>3 | | | |
| 11 | 5′CGCAGTCAGCC3′ <400>1 | Mismatches G—A & T—C | 501 | 0.310 |
| | 3′GCGTACGTCGG5′ <400>9 | | | |
| 4 | 5′CGCAGTCAGCC3′ <400>1 | Matches G—C & T—A | 500 | 0.196 |
| | 3′GCGTCAGTCGG5′ <400>3 | | | |
| 12 | 5′CACGCATTGCCCGCGTTGCGCA3′ <400>10 | Mismatch C—T | 504 | 0.315 |
| | 3′GTGCGTAACGTGCGCAACGCGT5′ <400>11 | | | |
| 13 | 5′CACGCATTGCCCGCGTTGCGCA3′ <400>10 | Match C—G | 507 | 0.260 |
| | 3′GTGCGTAACGGGCGCAACGCGT5′ <400>12 | | | |

Homo and Heteroduplexes (20 nmol) were incubated with KMnO₄ (100 nmol) in 1 ml of 3 M TEAC solutions 25° C.
^{a}Level of MnO₂ (arbitrary unit) was based on the absorbance at 420 nm.

### Example 4

Table 4 describes the protocol of the Mismatch Oxidation Colour (MOC) test for the model DNA samples 4 & 5. In brief, the set of DNA samples (20 nmol in tubes 1 & 2 or 40 nmol in tubes 4 & 5 of homo and heteroduplexes) were incubated with 10 μl or 20 μl of KMnO₄ respectively in volumes of 1 ml of 3M TEAC solution. The reaction mixtures as well as the control (no DNA in tube 3 & 6) were incubated at 25° C. for 1 h. The heteroduplex samples displayed strong yellow color in both concentration conditions while the homoduplex samples were pinkish (yellow/pink) compared to the control (strong pink). The yellow color development indicated higher level of MnO₂ in the heteroduplex reactions. The difference in colour between the homoduplex and heteroduplex samples were observed in all cases (4 to 13).

TABLE 4
Protocol for the MOC Test

| | | | KMnO₄ | 3M | |
|---|---|---|---|---|---|
| | Heteroduplex | Homoduplex | (10 nmol/ | TEAC | Time/ |
| Tube # | (0.5 nmol/μl) | (0.5 nmol/μl) | μl) | solution | Temp |
| 1 | 40 μl | — | 10 μl | 950 μl | 1 h/25° C. |
| 2 | — | 40 μl | 10 μl | 950 μl | 1 h/25° C. |
| 3 | — | — | 10 μl | 990 μl | 1 h/25° C. |
| 4 | 80 μl | — | 20 μl | 900 μl | 1 h/25° C. |
| 5 | — | 80 μl | 20 μl | 900 μl | 1 h/25° C. |
| 6 | — | — | 20 μl | 980 μl | 1 h/25° C. |

### Example 5

The isosbestic points of calf thymus DNA and the 547 bp mouse promoter (as above) were determined and compared by incubating 25 μg calf thymus DNA or 12.4 μg mouse promoter with 0.2 μmol of KMnO₄ in 1 ml of TEAC at 25° C. The results are depicted in Table 5.

TABLE 5
Comparative isosbestic points for calf thymus
DNA and mouse β-globin promoter

| | Isosbestic point |
|---|---|
| DNA duplexes | at 25° C. |
| calf thymus DNA | 495 nm |
| Mouse promoter | 492 nm |

### Example 6

The permanganate oxidation reactions performed on duplexes 4 and 5 at 25° C. (see Table 1, EXAMPLE 1) were repeated at 50° C. (Table 6 and FIG. 1).

TABLE 6
Percentage completion of permanganate oxidation
with two model duplexes 4 and 5 at 50° C.

| Time of Reaction | % reaction at 50° |
|---|---|
| min | Duplex 4/Duplex 5 |
| 5 | 37%/64% |
| 10 | 65%/78% |
| 15 | 72%/95% |
| 20 | 80%/96% |
| 25 | 88%/100% |
| 30 | 92%/100% |

At this higher temperature, the rate of oxidation, as measured by MnO₂ formation, was significantly faster than at 25° C., and it is noted that even after 5 min, the extent of oxidation of the homoduplex 4 was greater than that for duplex 4 at 25° C. after 30 min. Without being limited by theory, it is presumed that at 50° C., both duplexes are denatured and the rate of reaction is controlled by the individual number of T and C bases with each single stranded nucleic acid molecule. (Melting temperatures of 4 and 5 have been reported to be 50° C. and 45° C. respectively¹). Thus even at temperatures at or above the melting temperature of the duplexes, the oxidative reaction can be useful in identifying differences between two nucleic acid duplexes.

### Example 7

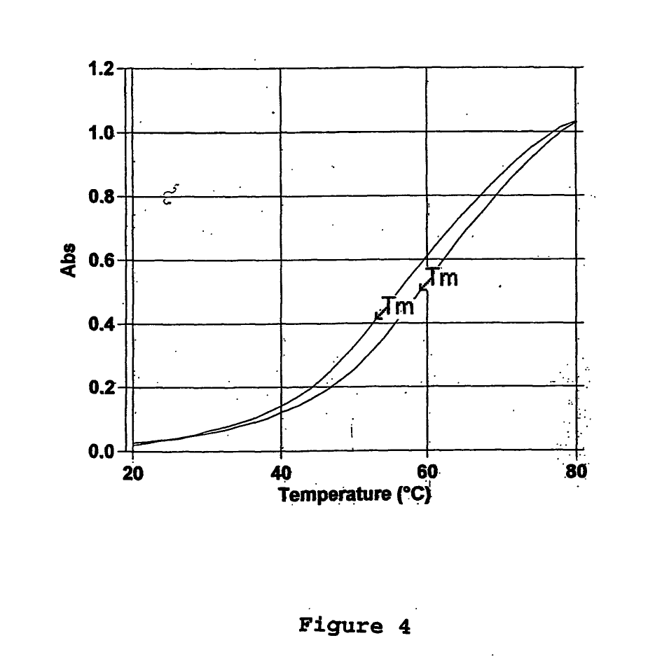

Two model heteroduplex DNA (22 bp and 38 bp, 20 nmol each) containing the T-C mismatches and their corresponding homoduplex DNA were allowed to react with KMnO₄ (20 μl, 0.2 μmol) in 1 ml of 3M TEAC solution. The reaction mixtures were heated in 1.2 ml quartz cuvettes from 20° C. to 80° C. at a rate of 2° C. per minute. The reactions were followed by measuring absorbance at 420 nm and the thermal analysis spectra and the results are displayed in the FIGS. 4 & 5 and Table 7 respectively.

The initial oxidation temperature can be readily obtained from the first derivative spectrophotometric method (Varian, Cary-300 Spectrophotometer). The results show that the initial oxidation temperature of the heteroduplex DNA is lower than that of the homoduplex DNA.

TABLE 7

Thermal analysis data for
permanganate oxidation of the model DNA

| | | Mismatch | Tm (initial | Level of |
|---|---|---|---|---|
| | | or match | oxidation | MnO₂ |
| # | Sequences | (control) | temp)^{c} | at 55° C^{d} |
| 12 | 5′CACGCATTGCCCGCGTTGCGCA3′ | Mismatch C-T | 53 | 0.468 |
| | 3′GTGCGTAACGTGCGCAACGCGT5′ | | | |
| 13 | 5′CACGCATTGCCCGCGTTGCGCA3′ | Match C-G | 59 | 0.380 |
| | 3′GTGCGTAACGGGCGCAACGCGT5′ | | | |
| 14 | DNA 17^{a} (38 bp) | Mismatch T-C | 51 | 0.752 |
| 15 | DNA 18^{b} (38 bp) | Match C-G | 55 | 0.625 |

^{a}DNA 14:
5′GGAAGAAGGCATACGGGTTAACTAGGGCAGCGGACAAT3′ <400>13
3′CCTTC T TCCG TATGCCCACTTGATCCC GTC GCCTGTTA5′ <400>14
^{b}DNA 15:
5′GGAAGAAGGCATACGGGTGAACTAGGGCAGCGGACAAT3′ <400>15
3′CCT TC T TCCG TATGCCCACTTGATCCC GTC GCCTGTTA5′ <400>14
^{c}Tm (initial oxidation temperature) were obtained by 1^{st} derivative melting calculation software (Cary-300 Spectrophotometer)
^{d}Level of MnO₂ (arbitrary unit) was based on the absorbance at 420 nm.

### Example 8

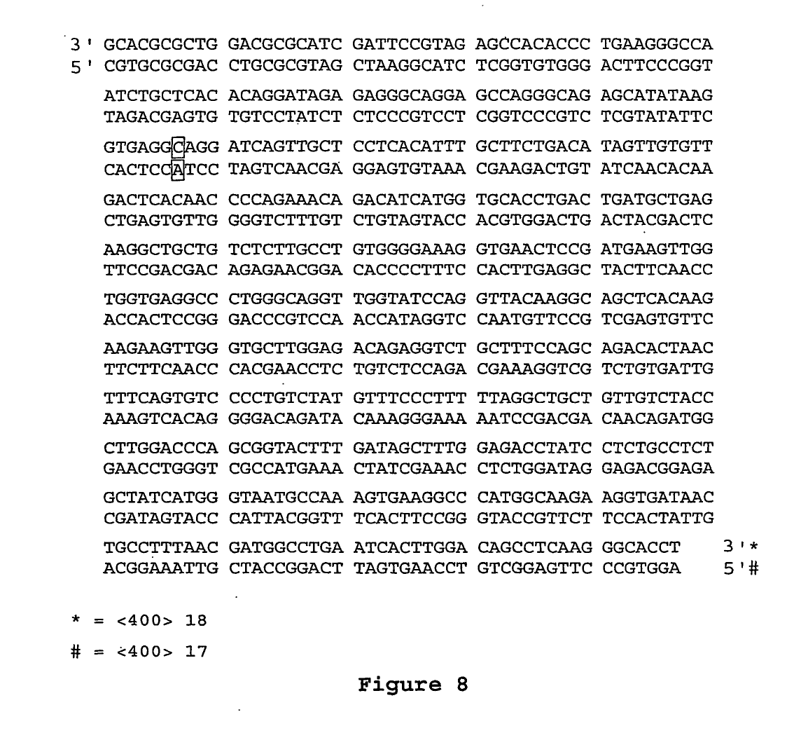

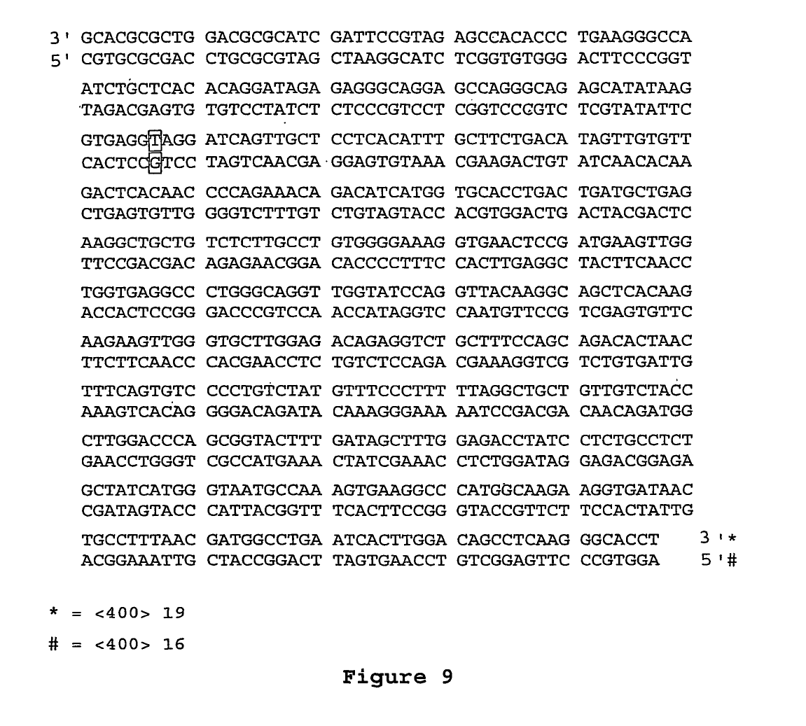

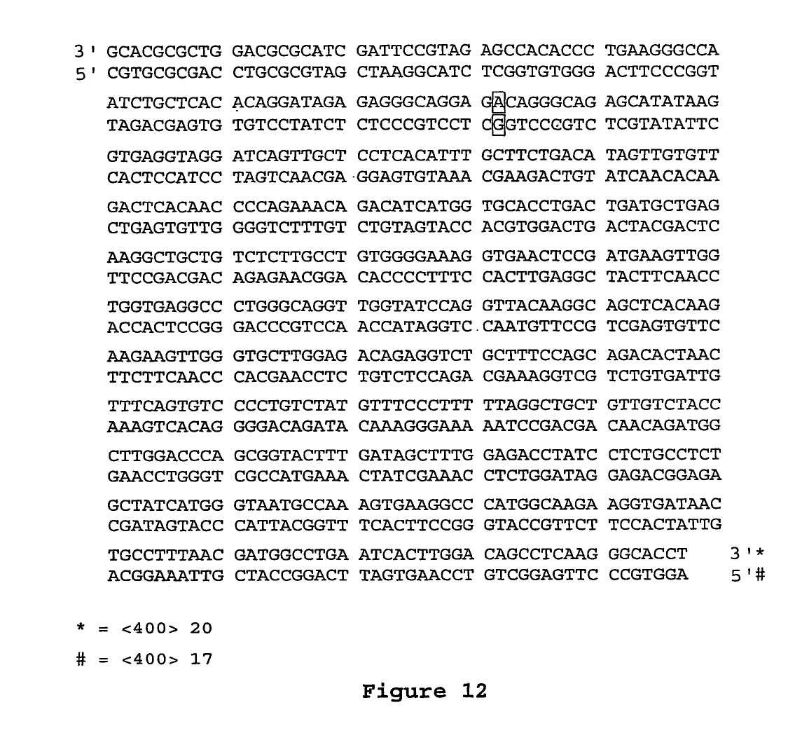

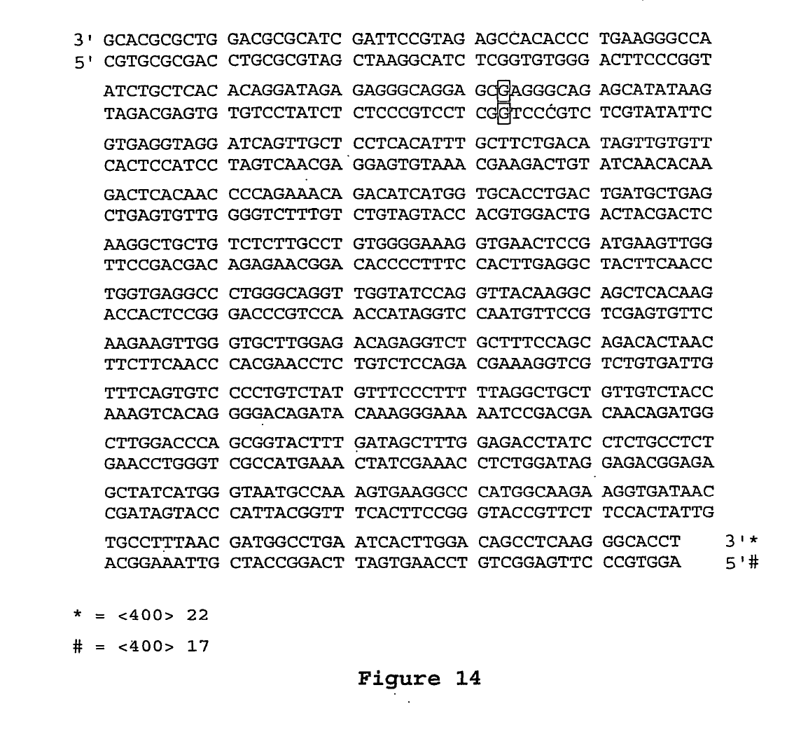

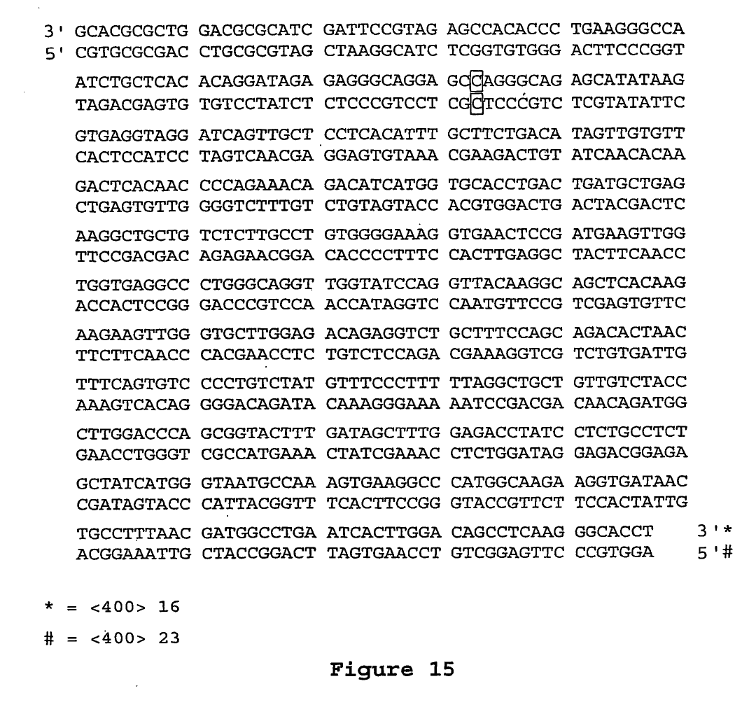

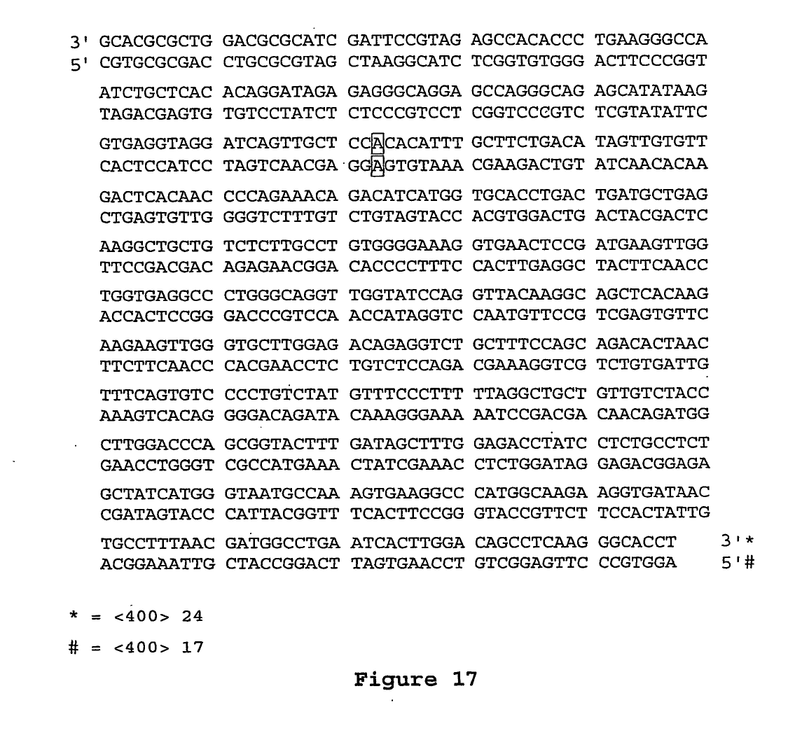

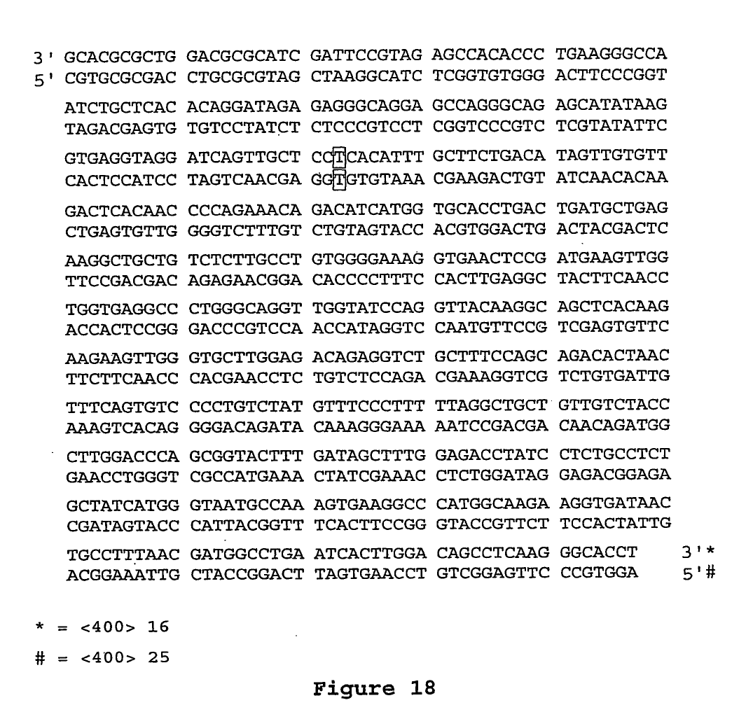

The following protocol (Table 8) has been developed for detection of longer mismatched DNA sequences. This condition was successfully applied to 547 bp DNA (β-globin mouse promoter) to detect all possible classes of mismatched base pair sets (C-A, T-G; C-T, A-G; G-G, C-C and A-A, T-T—FIGS. 8, 9, 11, 12, 14, 15, 17 and 18 respectively). Homoduplexes (wildtype and mutant) were obtained as previously described². Heteroduplex samples 16-19 were obtained by amplifying and mixing the wildtype and mutant homoduplexes.

TABLE 8
The Protocol Used for Detection of Long
Mismatched DNA Sequences (547 bp).

| DNA | KMnO₄ | | | Temp gradient |
|---|---|---|---|---|
| 2.0-2.5 μg/μl | (10 nmol/μl) | H₂O | TEAC solution* | (2° C./min) |
| 10 μl | 30 μl | 60 μl | 900 μl of 2M | 20 to 50 or 60° C. |
| | | | or 3M TEAC | |

*3M TEAC solution was used for the mismatch sets: T.G, A.C; T.C, A.G; C.C, G.G and 2M TEAC solution was used for the mismatch set: T.T, A.A to maximize the differences between homo and heteroduplexes

Thus, the DNA samples (547 bp fragments, Table 9) were incubated with KMnO₄ in 2M or 3M TEAC solutions. The reaction mixtures were initiated at 20° C. and then slowly increased up to 50° C. or 60° C. at the rate of 2° C./min. The oxidation levels were followed by measurement of the absorbance at 420 nm and the data was analyzed by absorbance at 420 nm,

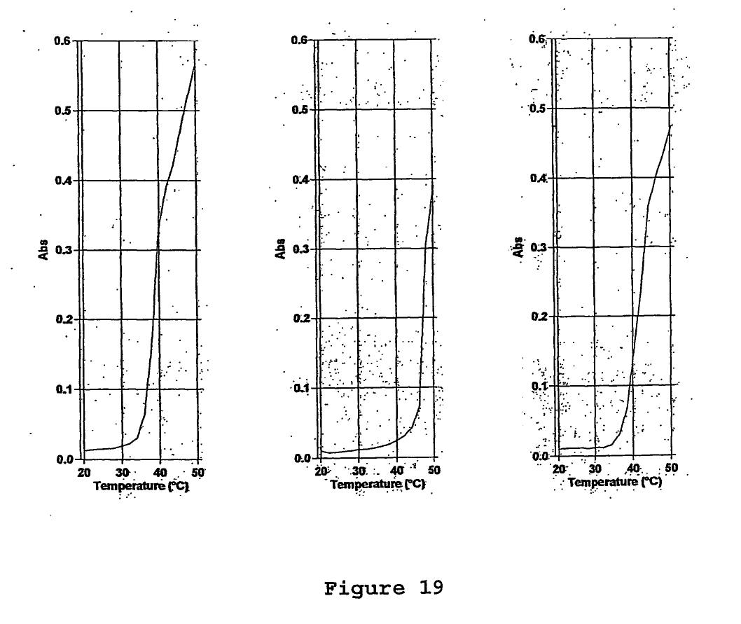

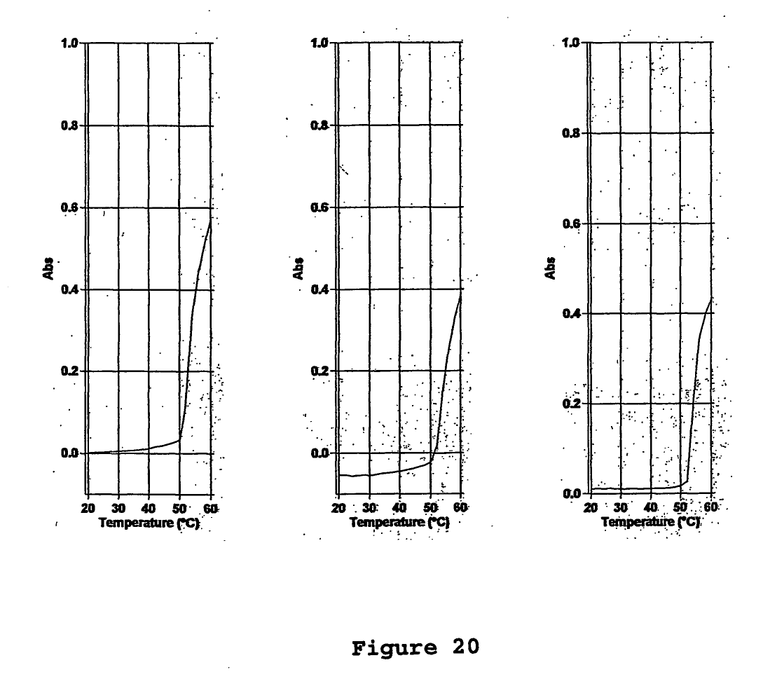

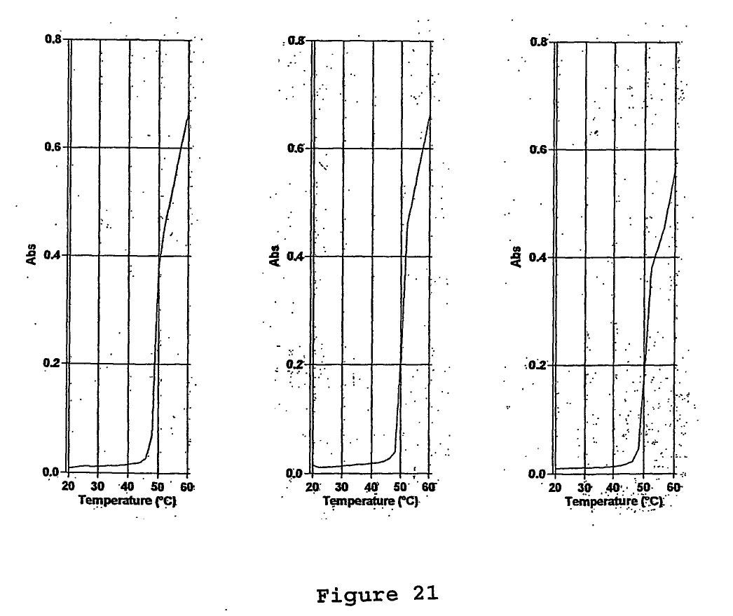

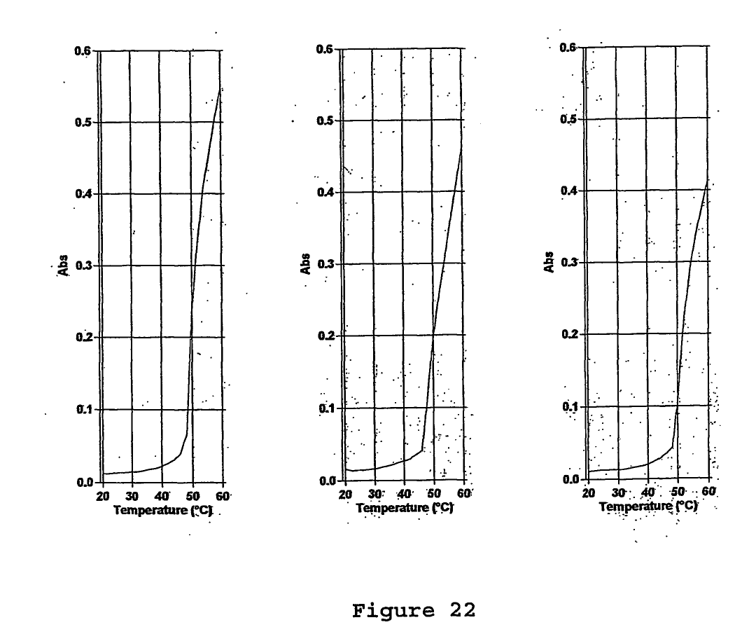

The oxidation reactions were initiated at different temperatures depending on DNA sequences (ie. homoduplex and heteroduplex DNA) as well as the mismatch types. In all cases, the oxidation levels of the heteroduplexes were higher than the corresponding homoduplexes (Table 9). The thermal analysis spectra of all 4 sets of mismatches are illustrated in FIGS. 19-22.

In order to enhance the resolution of the oxidation patterns obtained from the above experiments, UV-Visible derivative spectroscopy was employed as an analytical tool. The derivative results summarized in Table 10. In all cases of mismatch, the heteroduplexes exhibited their strongest signals with respect to 3^{rd} derivative spectroscopy compared to the homoduplexes DNA.

TABLE 9
Mutation Detection Based on the Absorbance
at 420 nm at optimal temperature^{a}

| | | Optimal | | | |
|---|---|---|---|---|---|
| Sample id | Mismatch | temp^{a} | Het | Homo (Wt) | Homo (Mt) |
| (mutation) | set | ° C. | A420 | A420 | A420 |
| 16 | T.G, C.A | 40 | 0.33 | 0.02 | 0.13 |
| 17 | C.T, A.G | 60 | 0.57 | 0.38 | 0.43 |
| 18 | C.C, G.G | 50 | 0.38 | 0.20 | 0.17 |
| 19 | T.T, A.A | 60 | 0.55 | 0.45 | 0.41 |

Note:
2M TEAC solution was applied for the last mismatch set (T.T, A.A). Similar trends and differences of the oxidation levels were observed in all repeated experiments. All experiments were carried out in duplicate.
^{a}Optimal temperature: the temperature gave maximum different absorbance at 420 nm between homoduplex and heteroduplex DNA

TABLE 10
UV-Visible 3rd Derivative Analysis of mismatched DNA samples

| | | Third derivative (arbitrary unit) | | |
|---|---|---|---|---|
| Sample id | Mismatch | Het | Homo (Wt) | Homo (Mt) |
| 16 | T.G, C.A | −0.042 (at 38° C.) | 0.000 (at 38° C.) | −0.005 (at 38° C.) |
| 17 | C.T, A.G | −0.060 (at 53° C.) | −0.030 (at 53° C.) | −0.040 (at 53° C.) |
| 18 | C.C, G.G | −0.125 (at 49° C.) | −0.020 (at 49° C.) | −0.015 (at 49° C.) |
| 19 | T.T, A.A | −0.058 (at 49° C.) | −0.002 (at 49° C.) | −0.003 (at 49° C.) |

### REFERENCES

1. John, D. M., and Weeks, K. M., Chemistry and Biology, 2000, 7, 405-410.

2. Lambrinakos, A., Humphrey, K. E., Babon, J. J., Ellis, T. P and Cotton, R. G. H., Nucleic Acids Research, 1999, 27, 1866.

1 A method for detecting a base pairing difference between a first nucleic acid molecule and a second nucleic acid molecule in a test nucleic acid duplex comprising the steps of:
(i) treating the test nucleic acid duplex with an effective amount of an oxidizing agent for a time and under conditions sufficient to oxidize a mismatched or unmatched base in the test nucleic acid duplex;
(ii) monitoring the formation, or rate thereof, of one or more reaction products; and/or the consumption, or rate thereof, of one or more starting agents; and
(iii) determining if there is a difference in the formation, or rate thereof, of one or more reaction products and/or the consumption, or rate thereof, of one or more starting agents between that of the test nucleic acid duplex and that of a control nucleic acid duplex which has separately been subjected to the same conditions of steps (i) and (ii);
wherein a difference in the formation, or rate thereof, of one or more reaction products and/or the consumption, or rate thereof, of one or more starting agents between the test and control nucleic acid duplexes is indicative of a base pairing difference between said first and second nucleic acid molecules in a test nucleic acid duplex.

2. A method according to claim 1 wherein the first and second nucleic acid molecules are nucleotide sequences.

3. A method according to claim 1 wherein the base pairing difference is the result of a point mutation, insertion or deletion in a nucleic acid molecule.

4. A method according to claim 1 wherein the mismatched or unmatched base in the test nucleic acid duplex is thymine, cytosine or uracil.

5. A method according to claim 4 wherein the mismatched or unmatched base in the test nucleic acid duplex is thymine or cytosine.

6. A method according to claim 1 wherein the monitoring of the formation, or rate thereof, of one or more reaction products and/or the consumption, or rate thereof, of one or more starting agents is by a technique selected from the group consisting of UV visible spectroscopy, NMR spectroscopy, mass spectroscopy, chromatography, titration, colorimetry, electrochemical detection, visual detection, melting temperature detection and redox stain.

7. A method according to claim 6 wherein the monitoring of the formation, or rate thereof, of one or more reaction products and/or the consumption, or rate thereof, of one or more starting agents involves monitoring for the presence of the oxidizing agent and/or the reduced form of the oxidizing agent.

8. A method according to claim 6 or 7 wherein the technique is UV visible spectroscopy.

9. A method according to claim 8 wherein the oxidizing agent is KMnO₄ and the monitoring of the formation, or rate thereof of a reaction product involves measuring absorbance at about 420 nm.

10. A method according to claim 8 wherein the oxidizing agent is KMnO₄ and the monitoring of the consumption, or rate thereof, of a starting agent involves measuring absorbance at about 525 nm.

11. A method according to claim 8 further comprising the determination of the isosbestic point for the oxidation of each of the test and control nucleic acid duplexes.

12. A method according to claim 6 wherein the technique is visual detection.

13. A method according to claim 6 wherein the technique involves monitoring for the melting temperature of the duplex undergoing treatment with the oxidizing agent.

14. A method according to claim 1 wherein the oxidizing agent is KMnO₄.

15. A method according to claim 14 wherein the treatment with KMnO₄ is carried out in the presence of TEAC or TMAC.

16. A kit adapted for performing the method of claim 1, wherein in said kit is in compartmentalized form and comprises at least two components selected from the group consisting of oxidizing agent, base (or salt thereof) test nucleic acid duplex or molecules, control nucleic acid duplex or molecules, buffer and spectroscopic cell.

17. A method according to claim 1 wherein a second nucleic acid duplex is simultaneously treated with said test nucleic acid duplex, said test and second nucleic acid duplexes being formed by mixing, melting and reannealing a control nucleic acid duplex and a test-mutant nucleic acid duplex.

18. A method for identifying a difference between a first nucleic acid duplex and a second nucleic acid duplex, comprising the steps of:
(i) separately treating each duplex with an oxidizing effective amount of an oxidizing agent for a time and under conditions sufficient to at least partially oxidize at least one duplex;
(ii) monitoring the formation, or rate thereof, of one or more reaction products and/or the formation, or rate thereof, of one or more starting agents for each duplex; and
(iii) determining if there is a difference in the formation, or rate thereof of one or more reaction products and/or the consumption, or rate thereof, of one or more starting agents between the first and second nucleic acid duplexes,
wherein a difference in the formation, or rate thereof, of one or more reaction products and/or the consumption, or rate thereof, of one or more starting agents is indicative of a difference between the first and second nucleic acid duplexes.

19. A method according to claim 18 wherein the monitoring of oxidation of each duplex is determined by a technique selected from the group consisting UV visible spectroscopy, NMR spectroscopy, mass spectroscopy, chromatography, titration, colorimetry, electrochemical detection, visual detection, melting temperature detection and redox stain.

20. A method according to claim 19 wherein the monitoring is determined by UV visible spectroscopy.

21. A method according to claim 20 wherein the oxidizing agent is KMnO₄ and the monitoring of the formation, or rate thereof, of a reaction product involves measuring absorbance at about 420 nm.

22. A method according to claim 20 wherein the oxidizing agent is KMnO₄ and the monitoring of the consumption, or rate thereof, of a starting agent involves measuring absorbance at about 525 nm.

23. A method according to claim 20 further comprising the determination of the isosbestic point for the oxidation of each duplex.

24. A method according to claim 19 wherein the technique involves monitoring for the melting temperature of the duplex undergoing treatment with the oxidizing agent

25. A method according to claim 18 wherein the oxidizing agent is KMnO₄.

26. A method according to claim 25 wherein the treatment with KMnO₄ is carried out in the presence of TEAC or TMAC.

25. A kit adapted for performing the method of claim 18, wherein in said kit is in compartmentalized form and comprises at least two components selected from the group consisting of oxidizing agent, base (or salt thereof) first and/or second nucleic acid duplexes or molecules for the formation thereof, buffer and spectroscopic cell.
